(19) United States
(12) Patent Application Publication
(10) Pub. No.: US 2022/0395531 A1
(43) Pub. Date: Dec. 15, 2022

(54) AEROSOLIZED COMPOSITIONS COMPRISING MITOCHONDRIA AND METHODS OF USE THEREOF

(71) Applicant: Children`s Medical Center Corporation, Boston, MA (US)

(72) Inventors: James D. McCully, Marblehead, MA (US); Pedro J. Del Nido, Lexington, MA (US)

(21) Appl. No.: 17/603,646

(22) PCT Filed: Apr. 15, 2020
(86) PCT No.: PCT/US2020/028219

§ 371 Date: Oct. 14, 2021

Related U.S. Application Data
(60) Provisional application No. 62/834,020, filed on Apr. 15, 2019.

(51) Int. Cl. A61K 35/00; A61P 11/00; A61K 9/00

(52) U.S. Cl. CPC A61K 35/00; A61P 11/00; A61K 9/0073

(57) ABSTRACT

This disclosure pertains to pharmaceutical compositions of aerosolized compositions containing mitochondria, methods of preparing and using the compositions, and devices for administering the compositions.

### CLAIM OF PRIORITY

This application is a U.S. National Phase Application under 35 U.S.C. § 371 of International Application No. PCT/US2020/028219, filed on Apr. 15, 2020, which claims the benefit of U.S. Provisional Application No. 62/834,020, filed on Apr. 15, 2019. The entire contents of the foregoing are incorporated herein by reference.

### SEQUENCE LISTING

This application contains a Sequence Listing that has been submitted electronically in ASCII format and is hereby incorporated by reference in its entirety. Said ASCII file is named SequenceListing.txt, created on Sep. 29, 2021, and is 1.1 kilobytes in size.

### TECHNICAL FIELD

This disclosure pertains to aerosolized compositions containing mitochondria, methods of preparing and using the compositions, and devices for administering the compositions.

### BACKGROUND

Mitochondria are double membrane-bound organelles found in the cytoplasm of nucleated eukaryotic cells. They are found in almost every cell of the human body except red blood cells. They are the cell's primary site of energy metabolism and generate adenosine triphosphate (ATP) for different cell functions. Typically, more than 90% of a cell's requirement for ATP is supplied by the cell's own mitochondria.

Mitochondria are composed of two concentric membranes, which have specialized functions. The inner mitochondrial membrane contains proteins for ATP synthase. The outer mitochondrial membrane, which contains large numbers of integral membrane proteins, encloses the entire organelle.

The structure of mitochondria has striking similarities to some modern prokaryotes. In fact, mitochondria are thought to have originated from an ancient symbiosis when a nucleated cell engulfed an aerobic prokaryote. In the symbiosis relationship, the host cell came to rely on the engulfed prokaryote for energy production, and the prokaryote cell began to rely on the protective environment provided by the host cell.

Due to mitochondria's primary function in cell metabolism, damage and dysfunction in mitochondria can cause a range of human diseases. Damage to mitochondria may be caused by injury, toxicity, chemotherapy, and age-related changes. Particularly, ischemia/reperfusion injury can cause mitochondrial damage, which will have a negative impact on oxygen consumption and energy synthesis. Treatments involving mitochondria can be particularly useful for mitochondria-related disorders. Some early treatments involve administering mitochondria by intra-muscular injection and intra-arterial injection. There is a need for administering mitochondria to various target sites of a subject through different administration routes for various purposes.

### SUMMARY

This disclosure provides compositions (e.g., aerosolized compositions) containing mitochondria, methods of preparing and using the compositions, and devices for administering the compositions. In one aspect, the disclosure provides methods of administering a composition containing mitochondria or combined mitochondrial agents through respiratory tract. In some embodiments, the composition is an aerosolized composition. In some embodiments, the composition is a liquid solution.

In one aspect, the disclosure relates to a method of treating a subject having a respiratory disorder, the method comprising administering a composition comprising a therapeutically effective amount of mitochondria to the subject through respiratory tract.

In some embodiments, the composition is an aerosolized composition. In some embodiments, the composition is converted into an aerosol form prior to administration to the subject by using a nebulizer, a vaporizer, a nasal sprayer, a pressurized metered dose inhaler, or a breath activated pressurized metered dose inhaler.

In some embodiments, the aerosol form of the composition comprises droplets that have a median size from 0.01 to 1000 microliters (e.g., from 0.1 to 1000 microliters, from 1 to 1000 microliters, from 0.1 to 100 microliters, from 0.1 to 500 microliters, or from 1 to 500 microliters). In some embodiments, the droplet has a size at least 0.01, 0.1, 0.5, 1, 2, 3, 4, 5, 6, 7, 8, 9, 10, 20, 30, 40, 50, 60, 70, 80, 90, 100, 200, 300, 400, 500, 600, 700, 800, 900, or 1000 microliters. In some embodiments, the droplet has a size less than 1, 2, 3, 4, 5, 6, 7, 8, 9, 10, 20, 30, 40, 50, 60, 70, 80, 90, 100, 200, 300, 400, 500, 600, 700, 800, 900, or 1000 microliters.

In some embodiments, the composition comprises respiration-competent mitochondria.

In some embodiments, the subject has respiratory failure, reduced respiratory function, lung inflammation, lung carcinoma, skin wrinkles, baldness, and/or cancer. In some embodiments, the subject has acute lung injury.

In some embodiments, the concentration of mitochondria in the composition is about 1×10⁴ to 5×10⁹ ml⁻¹ (e.g., about 1×10⁵ to 5×10⁸ ml⁻¹, 1×10⁶ to 5×10⁸ ml⁻¹, 1×10⁷ to 5×10⁸ ml⁻¹, 1×10⁵ to 1×10⁸ ml⁻¹, 1×10⁶ to 1×10⁸ ml⁻¹, 1×10⁷ to 1×10⁸ ml⁻¹, 1×10⁶ to 5×10⁷ ml⁻¹, 1×10⁶ to 1×10⁷ ml⁻¹, 1×10⁶ to 5×10⁷ ml⁻¹, 5×10⁶ to 1×10⁸ ml⁻¹). In some embodiments, the concentration is at least or about 1×10⁴ ml⁻¹, 5×10⁴ ml⁻¹, 1×10⁵ ml⁻¹, 5×10⁵ ml⁻¹, 1×10⁶ ml⁻¹, 5×10⁶ ml⁻¹, 1×10⁷ ml⁻¹, 5×10⁷ ml⁻¹, 1×10⁸ ml⁻¹, 5×10⁸ ml⁻¹, or 1×10⁹ ml⁻¹, 5×10⁹ ml⁻¹, or 1×10¹⁰ ml⁻¹. In some embodiments, the concentration is less than 1×10⁴ ml⁻¹, 5×10⁴ ml⁻¹, 1×10⁵ ml⁻¹, 5×10⁵ ml⁻¹, 1×10⁶ ml⁻¹, 5×10⁶ ml⁻¹, 1×10⁷ ml⁻¹, 5×10⁷ ml⁻¹, 1×10⁸ ml⁻¹, 5×10⁸ ml⁻¹, or 1×10⁹ ml⁻¹, 5×10⁹ ml⁻¹, or 1×10¹⁰ ml⁻¹.

In some embodiments, the subject is administered 1×10⁵ to 1×10⁹ of mitochondria per dose (e.g., 1×10⁵ to 5×10⁸, 1×10⁶ to 5×10⁸, 1×10⁷ to 5×10⁸, 1×10⁵ to 1×10⁸, 1×10⁶ to 1×10⁸, 1×10⁷ to 1×10⁸, 1×10⁶ to 5×10⁷, 1×10⁶ to 1×10⁷, 1×10⁶ to 5×10⁷, 5×10⁶ to 1×10⁸ mitochondria per dose). In some embodiments, the dose is at least 1×10⁴, 5×10⁴, 1×10⁵, 5×10⁵, 1×10⁶, 5×10⁶, 1×10⁷, 5×10⁷, 1×10⁸, 5×10⁸, or 1×10⁹, 5×10⁹, or 1×10¹⁰ mitochondria per dose. In some embodiments, the dose is less than 1×10⁴, 5×10⁴, 1×10⁵, 5×10⁵, 1×10⁶, 5×10⁶, 1×10⁷, 5×10⁷, 1×10⁸, 5×10⁸, or 1×10⁹, 5×10⁹, or 1×10¹⁰ mitochondria per dose.

In some embodiments, the mitochondria are autogenic, allogeneic, or xenogeneic.

In some embodiments, the composition further comprises a solution selected from the group consisting of: K⁺-HEPES with a pH from 7 to 8, saline, phosphate-buffered saline (PBS), serum, and plasma.

In some embodiments, the composition further comprises one or more osmolytes selected from the group consisting of: trehalose, sucrose, mannose, glycine, proline, glycerol, mannitol, sorbitol, betaine, and sarcosine.

In some embodiments, the composition further comprises a pharmaceutical agent. In some embodiments, the composition further comprises a pharmaceutically acceptable diluent, excipient, or carrier.

In some embodiments, the composition further comprises a therapeutic agent or a diagnostic agent, wherein the therapeutic agent or the diagnostic agent is linked to the mitochondria by a covalent bond, is embedded in the mitochondria, or is internalized within the mitochondria. In some embodiments, the therapeutic agent or the diagnostic agent is a therapeutic diagnostic agent. In some embodiments, the therapeutic agent or the diagnostic agent comprises an antibody or an antigen binding fragment thereof. In some embodiments, the therapeutic agent or the diagnostic agent is linked to the mitochondria by a covalent bond.

In some embodiments, the therapeutic agent or the diagnostic agent is embedded in or internalized within the mitochondria.

In some embodiments, the mitochondria are genetically modified.

In some embodiments, the mitochondria comprise exogenous polypeptides.

In some embodiments, the mitochondria comprise exogenous polynucleotides, DNA, RNA, mRNA, micro RNAs, nuclear RNAs, or siRNA.

In one aspect, the disclosure relates to an aerosolized composition comprising a plurality of liquid droplets comprising a buffer, wherein at least one liquid droplet in the plurality of liquid droplets comprises at least one mitochondrion.

In some embodiments, the buffer is selected from the group consisting of: K+-HEPES with a pH from 7 to 8, saline, phosphate-buffered saline (PBS), serum, and plasma.

In some embodiments, the composition further comprises one or more osmolytes selected from the group consisting of: trehalose, sucrose, mannose, glycine, proline, glycerol, mannitol, sorbitol, betaine, and sarcosine.

In some embodiments, the composition further comprises a pharmaceutical agent.

In some embodiments, the composition further comprises a pharmaceutically acceptable diluent, excipient, or carrier.

In some embodiments, the composition further comprises a therapeutic agent or a diagnostic agent, wherein the therapeutic agent or the diagnostic agent is linked to the mitochondrion by a covalent bond, is embedded in the mitochondrion, or is internalized within the mitochondrion.

In some embodiments, the mitochondrial agent is selected from the group consisting of a therapeutic agent, a chemotherapeutic agent, or a diagnostic agent.

In some embodiments, the mitochondrial agent comprises an antibody or an antigen binding fragment.

In some embodiments, the mitochondrial agent is linked to the mitochondrion by a covalent bond. In some embodiments, the mitochondrial agent is embedded in or internalized within the mitochondrion.

In some embodiments, the plurality of droplets have a median size from 1 to 1000 microliters.

In some embodiments, the mitochondrion are genetically modified.

In some embodiments, the mitochondrion comprise exogenous polypeptides.

In some embodiments, the mitochondrion comprise exogenous polynucleotides.

In some embodiments, the mitochondrion prior to administration to the subject are incubated with a composition comprising an enzyme.

In some embodiments, the composition comprises autogenic mitochondria, allogeneic mitochondria, xenogeneic mitochondria, or a mixture thereof.

In one aspect, the disclosure relates to a device for delivering mitochondria to a subject through respiratory tract, the device comprising: a housing; a reservoir disposed within the housing for a composition comprising mitochondria; an aerosol generator to produce an aerosol form of the composition; and an outlet through which the composition is delivered to the respiratory tract of the subject.

In some embodiments, the device is a nebulizer, a nasal sprayer, or an inhaler. In some embodiments, the aerosol generator is an ultrasonic nebulizer.

In some embodiments, the aerosol generator is a vibrating mesh device. In some embodiments, the aerosol generator comprises an air compressor, wherein the air compressor cause compressed air to flow through the composition and turn the composition into an aerosol form.

In one aspect, the disclosure provides device for delivering mitochondria to a subject through respiratory tract, the device comprising: a housing; a composition comprising mitochondria disposed within the housing; and an outlet configured to deliver the composition from the housing to the respiratory tract of the subject and to aerosolize the composition as the composition passes therethrough.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 is a schematic diagram showing a method of isolating mitochondria.

FIG. 2A is skeletal muscle tissue that was obtained using a biopsy punch.

FIG. 2B are microscopic images of isolated mitochondria under phase contrast illumination (bright field, BF) (left panel), under fluorescence labeled with MitoTracker Red CMXRos (middle panel), and the merged image (the right panel).

FIG. 2C is a plot that shows results for mitochondria yield per gram tissue wet weight.

FIG. 2D are transmission electron microscopy images of isolated mitochondria.

FIG. 2E is a bar graph that shows results of activity assays for the mitochondrial complex I-V.

FIG. 2F is a bar graph that shows the results for the state 3 (active) oxygen consumption (ADP-stimulated respiration).

FIG. 2G is a bar graph that shows results for the respiratory control index (RCI; state 3/state 4) for malate induced (complex I) and succinate induced (complex II) oxygen consumption in autologous mitochondria from pectoralis major.

FIG. 3A are overlaid microscope images showing transplanted mitochondria localized within cardiomyocytes.

FIG. 3B are overlaid microscope images showing transplanted mitochondria localized within cardiomyocytes.

FIG. 3C are overlaid microscope images showing transplanted mitochondria localized within cardiomyocytes

FIG. 3D are overlaid microscope images showing transplanted mitochondria localized within cardiomyocytes

FIG. 4A is an image that shows a representative transverse image obtained by positron emission tomography (PET) of rats administered ¹⁸F Rhodamine 6-G mitochondria (6×10⁷) delivered to the lungs by vascular infusion through the pulmonary artery.

FIG. 4B is an image that shows a representative coronal image obtained by PET of rats administered ¹⁸F Rhodamine 6-G mitochondria (6×10⁷) delivered to the lungs by vascular infusion through the pulmonary artery.

FIG. 4C is an image that shows a representative image of lungs obtained by PET of rats administered ¹⁸F Rhodamine 6-G mitochondria (6×10⁷) delivered to the lungs by vascular infusion through the pulmonary artery.

FIG. 5A is an image of immunohistochemical staining of mouse lung tissue following administration of exogenous mitochondria by a nebulizer.

FIG. 5B is an image of immunohistochemical staining of mouse lung tissue following administration of exogenous mitochondria by the pulmonary artery.

FIG. 6 are images of murine lungs following 2 hours acute lung injury (ALI) ischemia of the left lung and 24 hours recovery. The red circle indicates lungs that are subject to the ALI procedure. Right lung is indicted as R, the left lung is indicated as L. The heart (H) is shown in the middle.

FIG. 7A is a bar graph that shows lung resistance in the mouse lungs (n=4) subjected to 2 hours left lung ischemia following 24 hours recovery. Vehicle or Mitochondria were delivered intravascularly through the left pulmonary artery (V) or by nebulizer (N).

FIG. 7B is a bar graph that shows elastance in mouse lungs subjected to 2 hours left lung ischemia following 24 hours recovery. Vehicle or Mitochondria were delivered intravascularly through the left pulmonary artery (V) or by nebulizer (N).

FIG. 8A is a bar graph that shows quantification of circulating free mtDNA determined by real-time PCR on whole blood samples in BALB/cJ mice receiving respiration buffer only (Sham) or mitochondria (3×10⁷ of mitochondria in respiration buffer).

FIG. 8B are images of hematoxylin and eosin stained lung tissue.

FIG. 8C are images of Masson's trichrome stained lung tissue.

FIG. 8D are images of transmission electron microscopy of the lung tissues from the inferior right lobe.

FIG. 9A is an image that shows quantification of mitochondrial uptake and bio-distribution. PET-microCT imaging of ¹⁸F-Rhodamine 6G labeled mitochondria (1×10⁹) delivered to the lungs of Wistar rats both via injection to the pulmonary trunk.

FIG. 9B is an image that shows quantification of mitochondrial uptake and bio-distribution. PET-microCT imaging of 18F-Rhodamine 6G labeled mitochondria (1×10⁹) delivered to the lungs of Wistar rats as an aerosol via nebulization.

FIG. 10A is an immunohistochemical image (100×) demonstrating exogenous mitochondria from human cardiac fibroblast, delivered via vascular delivery being taken up in lung tissue.

FIG. 10B is an immunohistochemical image (100×) demonstrating exogenous mitochondria from human cardiac fibroblast, delivered via nebulization being taken up in lung tissue.

FIG. 11A is a bar graph that shows results of lung function analysis of dynamic compliance for lungs receiving mitochondria via vascular delivery.

FIG. 11B is a bar graph that shows results of lung function analysis of resistance for lungs receiving mitochondria via vascular delivery.

FIG. 11C is a bar graph that shows results of lung function analysis of tissue damping for lungs receiving mitochondria via vascular delivery.

FIG. 11D is a bar graph that shows results of lung function analysis of inspiratory capacity for lungs receiving mitochondria via vascular delivery.

FIG. 11E is a bar graph that shows results of lung function analysis of peak inspiratory pressure for lungs receiving mitochondria via vascular delivery.

FIG. 12A show results of lung function analysis of dynamic compliance for lungs receiving mitochondria via nebulization.

FIG. 12B is a bar graph that shows results of lung function analysis of resistance for lungs receiving mitochondria via nebulization.

FIG. 12C is a bar graph that shows results of lung function analysis of tissue damping for lungs receiving mitochondria via nebulization.

FIG. 12D is a bar graph that shows results of lung function analysis of inspiratory capacity for lungs receiving mitochondria via nebulization.

FIG. 12E is a bar graph that shows results of lung function analysis of peak inspiratory pressure for lungs receiving mitochondria via nebulization.

FIG. 13 shows results of lung function analysis, in particular representative pressure-volume loops.

FIG. 14A are images that show hematoxylin and eosin (H&E), myeloperoxidase staining (MPO), TUNEL, and transmission Electron Microscope (EM) images of lung tissue sections for lungs receiving vehicle via vascular delivery; scale bars: 500 μm.

FIG. 14B are images that show hematoxylin and eosin (H&E), myeloperoxidase staining (MPO), TUNEL, and transmission Electron Microscope (EM) images of lung tissue sections for lungs receiving mitochondria via vascular delivery; scale bars: 500 μm.

FIG. 14C are images that show hematoxylin and eosin (H&E), myeloperoxidase staining (MPO), TUNEL, and transmission Electron Microscope (EM) images of lung tissue sections for lungs receiving vehicle via nebulization; scale bars: 500 μm.

FIG. 14D are images that show hematoxylin and eosin (H&E), myeloperoxidase staining (MPO), TUNEL, and transmission Electron Microscope (EM) images of lung tissue sections for lungs receiving mitochondria via nebulization; scale bars: 500 μm.

FIG. 14E are images that show hematoxylin and eosin (H&E), myeloperoxidase staining (MPO), TUNEL, and transmission Electron Microscope (EM) images of lung tissue sections for the sham group; scale bars: 500 μm.

FIG. 15A is a bar graph showing severity of lung tissue injury at 24 hours of reperfusion for lungs receiving vehicle via vascular delivery (Vehicle V), mitochondria via vascular delivery (Mito V), and the sham group. Following 2 hours of ischemia and 24 hours of reperfusion, H&E stained sections (15×) were evaluated for severity of lung injury using previously described scoring system; the worse the tissue injury, the higher the score (1-5). Severity of tissue injury analysis show significantly decreased inflammatory cells infiltration and interstitial congestion with no signs of destruction of lung architecture in Mito V and Mito Neb lungs as compared to Vehicle V and Vehicle Neb.

FIG. 15B is a bar graph showing neutrophil count at 24 hours of reperfusion for lungs receiving vehicle via vascular delivery (Vehicle V), mitochondria via vascular delivery (Mito V), and the sham group. Following 2 hours of ischemia and 24 hours of reperfusion, H&E stained sections (15×) were evaluated for severity of lung injury using previously described scoring system; the worse the tissue injury, the higher the score (1-5). Severity of tissue injury analysis show significantly decreased inflammatory cells infiltration and interstitial congestion with no signs of destruction of lung architecture in Mito V and Mito Neb lungs as compared to Vehicle V and Vehicle Neb.

FIG. 15C is a bar graph showing TUNEL labeled nuclei per 100 nuclei at 24 hours of reperfusion for lungs receiving vehicle via vascular delivery (Vehicle V), mitochondria via vascular delivery (Mito V), and the sham group. Following 2 hours of ischemia and 24 hours of reperfusion, H&E stained sections (15×) were evaluated for severity of lung injury using previously described scoring system; the worse the tissue injury, the higher the score (1-5). Severity of tissue injury analysis show significantly decreased inflammatory cells infiltration and interstitial congestion with no signs of destruction of lung architecture in Mito V and Mito Neb lungs as compared to Vehicle V and Vehicle Neb.

FIG. 15D is a bar graph showing severity of lung tissue injury at 24 hours of reperfusion for lungs receiving vehicle via nebulization (Vehicle Neb), mitochondria via vascular delivery (Mito Neb), and the sham group. Following 2 hours of ischemia and 24 hours of reperfusion, H&E stained sections (15×) were evaluated for severity of lung injury using previously described scoring system; the worse the tissue injury, the higher the score (1-5). Severity of tissue injury analysis show significantly decreased inflammatory cells infiltration and interstitial congestion with no signs of destruction of lung architecture in Mito V and Mito Neb lungs as compared to Vehicle V and Vehicle Neb.

FIG. 15E is a bar graph showing neutrophil count at 24 hours of reperfusion for lungs receiving vehicle via nebulization (Vehicle Neb), mitochondria via vascular delivery (Mito Neb), and the sham group. Following 2 hours of ischemia and 24 hours of reperfusion, H&E stained sections (15×) were evaluated for severity of lung injury using previously described scoring system; the worse the tissue injury, the higher the score (1-5). Severity of tissue injury analysis show significantly decreased inflammatory cells infiltration and interstitial congestion with no signs of destruction of lung architecture in Mito V and Mito Neb lungs as compared to Vehicle V and Vehicle Neb.

FIG. 15F is a bar graph showing TUNEL labeled nuclei per 100 nuclei at 24 hours of reperfusion for lungs receiving vehicle via nebulization (Vehicle Neb), mitochondria via vascular delivery (Mito Neb), and the sham group. Following 2 hours of ischemia and 24 hours of reperfusion, H&E stained sections (15×) were evaluated for severity of lung injury using previously described scoring system; the worse the tissue injury, the higher the score (1-5). Severity of tissue injury analysis show significantly decreased inflammatory cells infiltration and interstitial congestion with no signs of destruction of lung architecture in Mito V and Mito Neb lungs as compared to Vehicle V and Vehicle Neb.

FIG. 15G is a bar graph showing wet to dry weight ratio in percent at 24 hours of reperfusion for lungs receiving vehicle via vascular delivery (Vehicle V), mitochondria via vascular delivery (Mito V), vehicle via nebulization (Vehicle Neb), mitochondria via vascular delivery (Mito Neb), and the sham group. Following 2 hours of ischemia and 24 hours of reperfusion, H&E stained sections (15×) were evaluated for severity of lung injury using previously described scoring system; the worse the tissue injury, the higher the score (1-5). Severity of tissue injury analysis show significantly decreased inflammatory cells infiltration and interstitial congestion with no signs of destruction of lung architecture in Mito V and Mito Neb lungs as compared to Vehicle V and Vehicle Neb.

FIG. 16 show quantified results from BAL cytokine analysis; all values are expressed as mean±SEM. n=4 for all groups.

### DETAILED DESCRIPTION

This present disclosure relates to compositions containing mitochondria (e.g., viable and functional mitochondria), methods of preparing and using the compositions, and devices for administering the compositions to the respiratory tract of a subject. The present disclosure shows that mitochondria can be effectively administered to the subject through respiratory tract, and delivering the aerosolized form of the composition comprising mitochondria to respiratory tract is unexpectedly as effective as intra-artery administration.

The present disclosure also provides methods that can safely and reliably generate pharmaceutical compositions comprising viable and functional mitochondria. The pharmaceutical compositions as described herein can have increased therapeutic value, increased commercial value, and lower production cost per therapeutic dose.

In one aspect, the present disclosure provides methods of delivering mitochondria or combined mitochondrial agents to cells lining and surrounding the respiratory tract. Such pharmaceutical compositions can be taken up by cells, and once localized intracellularly, the mitochondria are able to confer one or more biological effects. These biological effects include, but are not limited to, producing the energy for the cell through respiration, producing ATP (i.e., converting ADP to ATP), regulating cellular metabolism, initiating citric acid cycle or the Krebs cycle, producing heat, storing calcium ions, signaling through mitochondrial reactive oxygen species, regulating the membrane potential, inducing apoptosis-programmed cell death, engaging in calcium signaling (e.g., calcium-evoked apoptosis), synthesizing heme and/or steroid, and initiating hormonal signaling, etc.

### Aerosolized Compositions

The present disclosure provides aerosolized compositions comprising isolated mitochondria and/or combined mitochondrial agents. The present disclosure shows that cell organelles (e.g., mitochondria) in an aerosolized composition or in the form of aerosol can remain viable and functional, and thus can be effectively administered to the respiratory tract of a subject.

As used herein, the term “aerosol” refers to a suspension of fine particles or droplets in a carrier gas. As used herein, the term “aerosolization” refers to the process or the act of converting a composition into the form of an aerosol. The aerosolized composition comprises a plurality of particles (e.g., droplets, solid particles, liquid droplets, or hydrogel particles). In some embodiments, the particle has a size (e.g., a diameter) of about or less than 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, or 1000 μm. In some embodiments, the particle has a size (e.g., a diameter) of about or greater than 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, or 1000 μm. In some embodiments, the particle has a size (e.g., a diameter) of from 500 nm to 10 μm, from 500 nm to 100 μm, from 1 μm to 10 μm, from 1 μm to 100 μm, from 5 μm to 100 μm, from 10 μm to 100 μm, or from 1 μm to 500 μm. As used herein, the term “diameter” refers to the length of the longest straight line segment whose endpoints both lie on the surface of a particle.

In some embodiments, the aerosolized composition comprises particles (e.g., droplets) with different sizes. In some embodiments, about or at least 10%, 20%, 30%, 40%, 50%, 60%, 70%, 80%, 90%, 91%, 92%, 93%, 94%, 95%, 96%, 97%, 98%, or 99% of particles are from 500 nm to 10 μm, from 500 nm to 100 μm, from 1 μm to 10 μm, from 1 μm to 100 μm, from 5 μm to 100 μm, from 10 μm to 100 μm, or from 1 μm to 500 μm.

In some embodiments, the particles in the aerosolized composition can have a median size (e.g., a median diameter) of about or less than 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, or 1000 μm. In some embodiments, the particles can have a median size (e.g., a median diameter) of about or greater than 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, or 1000 μm. In some embodiments, the particles can have a median size (e.g., a median diameter) of from 500 nm to 10 μm, from 500 nm to 100 μm, from 1 m to 10 μm, from 1 μm to 100 μm, from 5 μm to 100 μm, from 10 μm to 100 μm, or from 1 μm to 500 μm. As used herein, the term “median size” refers to the median size (e.g., diameter) as determined on a volume basis. Where the term median size is used, it is understood to describe the particle size that divides the population in half such that 50% of the population on a volume basis is greater than or less than this size.

The aerosolized compositions can be made by various devices known in the art. Exemplary devices that can convert the composition into an aerosol form prior to administration include, e.g., a nebulizer, a vaporizer, a nasal sprayer, an inhaler, a soft mist inhaler, a jet nebulizer, an ultrasonic wave nebulizer, a pressurized metered dose inhaler, a breath activated pressurized metered dose inhaler, or a vibrating mesh device. These devices can use various carrier gases, including e.g., oxygen, air, nitrogen, or compressed air.

The composition before aerosolization can include one or more pharmaceutically acceptable carriers. A pharmaceutically acceptable carrier can include a compound or composition useful in facilitating storage, stability, administration, cell targeting and/or delivery of the mitochondria and/or combined mitochondrial agent. The pharmaceutically acceptable carrier include, without limitation, suitable vehicles, diluents, solvents, excipients, pH modifiers, osmolytes, salts, colorants, rheology modifiers, lubricants, coatings, fillers, antifoaming agents, polymers, hydrogels, surfactants, emulsifiers, adjuvants, preservatives, phospholipids, fatty acids, mono-, di- and tri-glycerides and derivatives thereof, waxes, oils and water. In some embodiments, isolated mitochondria and/or the combined mitochondrial agents are suspended in water, saline, buffer, respiration buffer, or sterile mitochondria buffer for delivery in vivo. Pharmaceutically acceptable salts, buffers or buffer systems, including, without limitation, saline, phosphate buffer, phosphate buffered saline (PBS) or respiration buffer can be included in a composition described herein. Vehicles having the ability to facilitate delivery to a cell in vivo, such as liposomes, may be utilized to facilitate delivery of mitochondria or the combined mitochondrial agents to the target cells.

Exemplary buffers include, but are not limited to, a respiration buffer (e.g., 250 mmol/L sucrose, 20 mmol/L K+-HEPES buffer, pH 7.2, 0.5 mmol/L K+-EGTA, pH 8.0). Exemplary osmolytes include, but are not limited to, trehalose, sucrose, mannose, glycine, proline, glycerol, mannitol, sorbitol, betaine, and sarcosine.

### Isolated Mitochondria and Combined Mitochondrial Agents

Mitochondria for use in the presently described methods can be isolated or provided from any source, e.g., isolated from cultured cells or tissues. Exemplary cells include, but are not limited to, muscle tissue cells, cardiac fibroblasts, cultured cells, HeLa cells, prostate cancer cells, yeast, blood cells, cultured cells, and among others, and any mixture thereof. Exemplary tissues include, but are not limited to, liver tissue, skeletal muscle, heart, brain, blood, and adipose tissue (e.g., brown adipose tissue). Mitochondria can be isolated from cells of an autogenous source, an allogeneic source, and/or a xenogeneic source. In some instances, mitochondria are isolated from cells with a genetic modification, e.g., cells with modified mtDNA or modified nuclear DNA.

Mitochondria can be isolated from cells or tissues by methods known to those of skill in the art. In some embodiments, tissue samples or cell samples are collected and then homogenized. Following homogenization, mitochondria are isolated by repetitive centrifugation. Alternatively, the cell homogenate can be filtered through nylon mesh filters. Typical methods of isolating mitochondria are described, for example, in McCully et al., Injection of isolated mitochondria during early reperfusion for cardioprotection, Am J Physiol 296, H94-H105. PMC2637784 (2009); Frezza et al., Organelle isolation: functional mitochondria from mouse liver, muscle and cultured fibroblasts. Nature protocols, 2(2), 287-295 (2007); US20180057610; and US20180057610A1; each of which is incorporated by reference.

The mitochondria for the treatment can be isolated from cells or tissues of an autogenous source, an allogeneic source, and a xenogeneic source. In some instances, mitochondria are collected from cultured cells or tissues of a subject, and these mitochondria are administered back to the same subject. In some other cases, mitochondria are collected from cultured cells or tissues of a second subject, and these mitochondria are administered to a first subject. In some cases, mitochondria are collected from cultured cells or tissues from a different species (e.g., mice, swine, yeast).

The present disclosure also provides a composition comprising combined mitochondrial agent. The combined mitochondrial agents include e.g., mitochondria that are physically associated with an agent, such as a therapeutic agent, a diagnostic agent, and/or an imaging agent.

A therapeutic agent can be any agent that has a therapeutic or prophylactic use. Exemplary therapeutic agents include, e.g., therapeutic agents for ischemia-related disorders, cytotoxic agents for treating cancer, among many others. In some instances, mitochondria can deliver therapeutic agents to specific cells, for example, tumor cells. The therapeutic agent may be, e.g., an intracellular inhibitor, deactivator, toxin, arresting substance and/or cytostatic/cytotoxic substance that, once inside a cell, inhibits, destroys, arrests, modifies and/or alters the cell such that it can no longer function normally and/or survive. The therapeutic agent can be an agent to restore a cell's proper function, for example, a DNA vector for gene therapy. A therapeutic agent can be, e.g., an inorganic or organic compound; a small molecule (less than 500 daltons) or a large molecule; a proteinaceous molecule, such as a peptide, polypeptide, protein, post-translationally modified protein, or antibody; or a nucleic acid molecule, such as a double-stranded DNA, single-stranded DNA, double-stranded RNA, single-stranded RNA, or a triple helix nucleic acid molecule. In some embodiments, a therapeutic agent can be a natural product derived from any known organism (e.g., from an animal, plant, bacterium, fungus, protist, or virus) or from a library of synthetic molecules. In some embodiments, a therapeutic agent can be a monomeric or a polymeric compound. Some exemplary therapeutic agents include cytotoxic agents, DNA vectors, small interfering RNAs (siRNA), micro RNAs (miRNA), reactive peptides, nanoparticles, microspheres, and fluorescent molecules.

A diagnostic agent is an agent that has diagnostic use. As mitochondria carry a diagnostic agent into a cell, in some embodiments, the diagnostic agent can be designed to determine the condition within a cell, for example pH and oxidative stress within a cell.

An imaging agent is an agent that is employed for use in imaging techniques. The techniques or modalities include, but are not limited to, X-rays, computed tomography (CT), magnetic resonance imaging (MRI), scintigraphy, fluorescence, ultrasound, etc. The imaging agent can be florescent and/or radioactive. In some embodiments, an imaging agent can also be a diagnostic agent. Exemplary imaging agents include, but are not limited to, MitoTracker fluorophores (Thermo Fisher Scientific Inc.), CellLight® RFP, BacMam 2.0 (Thermo Fisher Scientific Inc.), pH-sensitive pHrodo fluorescent dyes (Thermo Fisher Scientific Inc.), ¹⁸F-Rhodamine 6G, ¹⁸F-labeled rhodamine B, magnetic iron oxide nanoparticles, and gold- and platinum-based nanoparticles.

As discussed above, a combined mitochondrial agent comprises a mitochondria and an agent that are in direct and/or indirect physical contact with each other. For example, an agent can be linked to mitochondria, attached to mitochondria, embedded in the mitochondrial membrane, or completely or partially enclosed in mitochondria. In some instances, a pharmaceutical agent can be linked to mitochondria covalently. In some instances, the agent is linked to constituents of mitochondrial membrane directly through a covalent bond (e.g., a carboxamide bond and a disulfide bond), or indirectly through a linker (e.g., a peptide linker) or another covalently bonded agent. In other instances, an agent can be linked to mitochondria non-covalently, for example, through hydrophobic interaction, Van der Waals interaction, and/or electrostatic interaction, etc.

In some embodiments, a combined mitochondrial agent can comprise two or more different types of agents, for example, two different kinds of therapeutic agents, three different kinds of imaging agents, one therapeutic agent and one imaging agent, a therapeutic agent and a diagnostic agent, etc. Skilled practitioner will appreciate that any variation is possible.

One particularly useful linker to link mitochondria and an agent provides a sustained release of the agent upon injection. This can be accomplished, for example, using a hydrazone functional group. For example, a hydrazone is formed to covalently bind an agent to constituents on the mitochondrial membrane. Once this combined mitochondrial agent is taken up by cells, the change in pH will result in hydrolysis of the hydrazone, releasing the bound agent inside the cell.

In some embodiments, a therapeutic agent, a diagnostic agent, and/or an imaging agent can be linked to the outer mitochondrial membrane using functionalized surface chemistry. In some cases, heterobifunctional chemistries can link a therapeutic agent, a diagnostic agent, and/or an imaging agent to the mitochondrial surface, and once they are internalized, these agents can be released through interactions with intercellular esterases (e.g. via interaction with an acetoxymethyl ester) or through a UV-light activation or Near-Infrared light activation strategy. The UV-light activation and Near-Infrared light activation strategies are described, e.g., in Zhou et al., “Progress in the Field of Constructing Near-Infrared Light-Responsive Drug Delivery Platforms,” Journal of Nanoscience and Nanotechnology 16.3 (2016): 2111-2125; Bansal et al., “Photocontrolled nanoparticle delivery systems for biomedical applications,” Accounts of chemical research 47.10 (2014): 3052-3060; Barhoumi et al., “Ultraviolet light-mediated drug delivery: Principles, applications, and challenges,” Journal of Controlled Release 219 (2015): 31-42; and US20180057610A1. Each of them is incorporated by reference in its entirety.

Skilled practitioners will appreciate that in some instances a composition described herein may include more than one type of combined mitochondrial agent. For example, included are compositions comprising mitochondria wherein essentially each mitochondrion is associated with multiple types of agents. Also included are compositions comprising mitochondria wherein each mitochondrion is paired with only one type of agent but wherein the composition comprises a mixture of mitochondria/agent pairings.

### Methods of Making Combined Mitochondrial Agents

Skilled practitioners will appreciate that an agent can be linked to mitochondria in any number of ways, e.g., by attaching to mitochondria, embedding partially or completely in the mitochondrial membrane, enclosing in mitochondria, or encapsulating within the mitochondria.

While not intending to be bound by any theory or any particular approach, it is believed that the outer membrane of mitochondria is adherent and thus particularly amenable to combination with various agents. In some embodiments, pharmaceutical agents can be attached to the outer membrane of mitochondria simply by incubation. For example, an effective amount of pharmaceutic agents can be fully mixed with isolated mitochondria in a buffer, e.g., respiration buffer, at a temperature favorable to isolated mitochondria, e.g., from 0° C. to 26° C., from 0° C. to 4° C., or about 0° C., 4° C., 26° C. This procedure is useful to attach an effective amount of pharmaceutic agents (e.g., nanoparticles, DNA vectors, RNA vectors) to mitochondria.

In some embodiments, organic cations (e.g., rhodamine and tetramethylrosamine) are readily sequestered by functioning mitochondria because of the electric potential on mitochondrial membrane. Healthy mitochondrial membranes maintain a difference in electric potential between the interior and exterior of the organelle, referred to as the membrane potential. This membrane potential is a direct result of mitochondrial functional processes, and can be lost if the mitochondria are not working properly. Lipid-soluble cations are sequestered by mitochondria as a consequence of their positive charge and of their solubility in both the inner membrane lipids and the matrix aqueous space. Similarly, in some other embodiments, anions can be attached to the outer membrane of mitochondria because of its negative charge. To link mitochondria with these pharmaceutical agents, an effective amount of pharmaceutic agents should be fully mixed with isolated mitochondria in a buffer, e.g., respiration buffer, at a temperature favorable to isolated mitochondria, e.g., about 0° C. or 4° C.

The therapeutic, diagnostic, and/or imaging agent can be linked to phospholipids, peptides, or proteins on the mitochondrial membrane through a chemical bond. For example, molecules including fluorophores (pHrodo Red (Thermo Fisher Scientific, Inc.)) and metallic particles (e.g., 30 nm magnetic iron oxide nanoparticles (Sigma)) can be covalently linked to exposed amine groups on proteins and peptides exposed on the outside membrane of intact mitochondria using succinimidyl ester conjugates. These reactive reagents react with non-protonated aliphatic amine groups, including the amine terminus of proteins and the ϵ-amino group of lysine residues, which creates a stable carboxamide bond. In another example, when the pharmaceutic agent, e.g., MitoTracker® Orange CMTMRos (Invitrogen, Carlsbad, Calif., now Thermo-Fisher Scientific, Cambridge, Mass.), are mixed with functional mitochondria, they are oxidized and then react with thiols on proteins and peptides on mitochondria to form conjugates.

There are numerous reactive chemical moieties available for attaching therapeutic, diagnostic, and/or imaging agents to the surface of mitochondria (e.g. carboxylic acid, amine functionalized, etc.).

Agents can be attached via protein bonding, amine bonding or other attachment methods either to the outer or inner mitochondrial membrane. Alternatively, or in addition, an agent can be attached to the mitochondria membrane through hydrophobic interaction, Van der Waals interaction, and/or electrostatic interaction.

In many instances, therapeutic agents, diagnostic agents and imaging agents may simply be mixed with isolated mitochondria, and incubated in a buffer (e.g., respiration buffer) for a sufficient period of time (e.g., a few minutes, 5 minutes, 10 minutes, or 1 hour) at favorable conditions (e.g., from 0° C. to 26° C., from 0° C. to 4° C., or about 0° C., 4° C., 26° C., pH 7.2˜8.0).

Exemplary methods of preparing combined mitochondrial agents are described in McCully et al, Injection of isolated mitochondria during early reperfusion for cardioprotection, Am J Physiol 296, H94-H105. PMC2637784 (2009); and Masuzawa et al, Transplantation of autologously derived mitochondria protects the heart from ischemia-reperfusion injury, Am J Physiol 304, H966-982. PMC3625892 (2013); and US20180057610A1. Each of the foregoing are incorporated by reference in its entirety.

### Methods of Treatment

The methods described herein include methods for the treatment of various diseases (e.g., lung disorders, respiratory disorders), ischemia reperfusion injury, and various other diseases described herein. Generally, the methods include administering a therapeutically effective amount of a composition comprising isolated mitochondria or combined mitochondria agent as described herein, to a subject who is in need of, or who has been determined to be in need of, such treatment. In some embodiments, the composition is administered through respiratory tract.

As used in this context, to “treat” means to ameliorate at least one symptom of the disorder (e.g., shortness of breath, fast breathing, low oxygen level in the blood, lung failure, respiratory failure, heart failure (e.g., right heart failure)). In some embodiments, the treatment results in improvement in pulmonary functions (e.g., increase of oxygen level in the blood, reducing resistance, reducing elastance). In some embodiments, the methods described herein can be used to treat pulmonary fibrosis, lung infection (e.g., by coronavirus), asthma, chronic obstructive pulmonary disease, pulmonary emboli, lung cancer, pulmonary hypertension, or lymphangioleiomyomatosis. In some embodiments, the methods described herein can be used to treat pneumonia.

The terms “subject” and “patient” are used interchangeably throughout the specification and describe an animal, human or non-human, to whom treatment according to the methods of the present invention is provided. Veterinary and non-veterinary applications are contemplated by the present invention.

A human patient can be an adult or a child, such as a newborn child. For example, the human subject can be at least or about 60 years old, e.g., at least about 65 years old, 70 years old, 75 years old, or at least or about 80 years old. The human subject can be below or about the age of 18 years old, e.g., 15 years old, 10 years old, 9 years old, 8 years old, 7 years old, 6 years old, 5 years old, 4 years old, 3 years old, 2 years old, or below or about 1 year old.

In addition to humans, subjects can include, but are not limited, non-humans, e.g., mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

Administering the compositions as described herein can be accomplished by various means, for example, intravenous, intradermal, subcutaneous, transdermal (topical), transmucosal, rectal administration. nasal administration, nasal instillation, insufflation (e.g., nasal sprays), inhalation (through nose or mouth), intrapulmonary administration, intratracheal administration, intraperitoneal injection, infusion, or any combinations thereof. In some embodiments, the composition is administered through respiratory tract.

In one aspect, the isolated mitochondria and/or combined mitochondrial agents can be delivered to lungs or heart to decrease stunning and allow for weaning of the organs from a surgical procedure (e.g., lung surgery or heart surgery). In some embodiments, the methods involve administering isolated mitochondria and/or combined mitochondrial agents through the respiratory tract.

In one aspect, the isolated mitochondria and/or combined mitochondrial agents can be delivered to ears, eyes, skin (e.g., for reducing wrinkles), or head (e.g., for baldness).

The compositions described herein can be administered to a subject as a singular, one-time treatment, or alternatively, multiple treatments, e.g., a treatment course that continues intermittently or continuously for about 1, 2, 5, 8, 10, 20, 30, 50, or 60 days, one year, indefinitely, or until a health care provider determines that administration of the mitochondria or combined mitochondrial agent is no longer necessary.

Isolated mitochondria and/or combined mitochondrial agents can be administered to a subject every as a single dose or multiple dosed delivered every 5 min or at 12-24 hours. In some embodiments, isolated mitochondria or combined mitochondrial agents can be administered to a subject every 5-10 minutes (e.g., every 5 minutes, every 10 minutes).

It is noted that in some cases, the effects of mitochondria depend on the length of the time period between the time of isolation and the time of use. Thus, in some instances, the mitochondria are freshly isolated and viable. The mitochondria or combined mitochondrial agents can be administered to a subject within about 5 minutes, about 10 minutes, about 20 minutes, about 30 minutes, about 40 minutes, about 50 minutes, about 60 minutes, about 70 minutes, about 80 minutes, about 90 minutes, about 100 minutes, about 110 minutes, about 120 minutes after the mitochondria are isolated. In some instances, the mitochondria or combined mitochondrial agents are administered to a subject within about 5 minutes, about 10 minutes, about 20 minutes, about 30 minutes, about 40 minutes, about 50 minutes, about 60 minutes, about 70 minutes, about 80 minutes, about 90 minutes, about 100 minutes, about 110 minutes, about 120 minutes after starting the mitochondria isolating process. Mitochondria and/or combined mitochondrial agents may in some instances be stored for a short period of time (e.g., about or at least 10 minutes, 20 minutes, 30 minutes, 40 minutes, 50 minutes, or 60 minutes) before use.

It is also noted that, in some cases, frozen-thawed mitochondria are not viable and not effective for certain treatments described herein, e.g., treatment of ischemia/reperfusion injuries. Thus, in some cases, the mitochondria are not frozen and thawed after isolation from tissues and/or cells.

In some embodiments, the compositions and the methods described herein can be used to treat disorders associated with lungs, ears, eyes, skin, scalp. For example, the compositions and the methods described herein can be used to treat cancers, wrinkle (e.g., wrinkles on skins), or baldness. In some cases, the compositions and the methods described herein to enhance the functions of various tissues and organs (e.g., lungs, ears, eyes, skin).

### Administration Through Respiratory Tract

The mucus (1-10 μm thick) that lines the pulmonary epithelium and the surfactant that lines the alveoli (0.1-0.2 μm thick) constitute physical barriers to pulmonary absorption of foreign particles. Furthermore, membrane-associated (epithelial and endothelial) and intracellular (macrophages, lymphocytes, neutrophils and mast cells) proteases and peptidases readily degrade administered peptides and proteins (Agu et al., “The lung as a route for systemic delivery of therapeutic proteins and peptides.” Respiratory research 2.4 (2001): 198). The pulmonary macrophages have also been shown to secrete or release short-lived peroxidases, inflammatory and immunomodulatory mediators (including granulocyte colony-stimulating factor, interleukins, leukotrienes and proteases), and other molecules as part of a host defense mechanism.

Nevertheless, the present disclosure shows that administering a composition comprising mitochondria or combined mitochondria agents through the respiratory tract can effectively promote lung function, reduce lung resistance, reduce lung elastance, reduce ischemic area, and/or reduce edema and cellular damage in the lungs.

The compositions as described herein can be administered through respiratory tract by various means, for example, nasal administration, nasal instillation, insufflation (e.g., nasal sprays), inhalation (through nose or mouth), intrapulmonary administration, intratracheal administration, or any combinations thereof. As used herein, the term “nasal instillation” refers to a procedure that delivers a therapeutic agent directly into the nose and onto the nasal membranes, wherein a portion of the therapeutic agent can pass through tracheas and is delivered into the lung.

Because of the occasionally limited functionally for the lungs that are in need of treatment, a therapeutic agent sometimes cannot be effectively delivered to the target sites in the lungs (e.g., bronchioles or alveoli) through respiratory tract administration. In these cases, an agent that can clear the airways can be administered to the subject first. In some embodiments, these agents can induce dilation of bronchial passages, and/or vasodilation in muscle. Such agents include, but are not limited to, beta2 adrenergic receptor agonists, anticholinergic agents, corticosteroids. In some embodiments, an agent for treating asthma can be used.

Pharmaceutical compositions suitable for administering through respiratory tract can include, e.g., liquid solutions, aqueous solutions (where water soluble), or dispersions, etc. In some embodiments, these compositions can comprise one or more surfactants.

As used herein, the term “respiratory tract” refers to the air passages from the nose to the pulmonary alveoli, including the nose, throat, pharynx, larynx, trachea, bronchi, and any part of the lungs. In some embodiments, the composition is administered to the lungs or any part of the respiratory system.

In some embodiments, the compositions can be administered a subject by a delivery system that can convert the composition into an aerosol form, e.g., a nebulizer, a vaporizer, a nasal sprayer, an inhaler, a soft mist inhaler, a jet nebulizer, an ultrasonic wave nebulizer, a pressurized metered dose inhaler, a breath activated pressurized metered dose inhaler, or a vibrating mesh device. As used herein, the term “inhaler” refers to a device for administering compositions in the form of a spray or dry powder that is inhaled (breathed in either naturally or mechanically forced in to the lungs) through the nose or mouth. In some embodiments, inhalers include e.g., a passive or active ventilator (mechanical with or without an endotracheal tube), nebulizer, dry powder inhaler, metered dose inhaler, and pressurized metered dose inhaler. Once mitochondria are deposited or localized near cells, a subset of the mitochondria can be taken up by the cells.

In some embodiments, the devices can use air (e.g., oxygen, compressed air) or ultrasonic power to break up solutions and suspensions into small aerosol particles (e.g., droplets) that can be directly inhaled from the mouthpiece of the device. In some embodiments, the devices use a mesh/membrane with laser drilled holes (e.g., from 1000 to 7000 holes) that vibrates at the top of the liquid reservoir, and thereby pressures out a mist of very fine droplets through the holes.

The delivery system can also have a unit dose delivery system. The volume of solution or suspension delivered per dose can be anywhere from about 5 to about 2000 microliters, from about 10 to about 1000 microliters, or from about 50 to about 500 microliters. Delivery systems for these various dosage forms can be dropper bottles, plastic squeeze units, atomizers, nebulizers or pharmaceutical aerosols in either unit dose or multiple dose packages.

In some embodiments, the device is a small, hard bottle to which a metered dose sprayer is attached. The metered dose can be delivered by drawing the composition into a chamber of defined volume, which chamber has an aperture dimensioned to aerosolize and aerosol formulation by forming a spray when a liquid in the chamber is compressed. The chamber is compressed to administer the composition. In certain devices, the chamber is a piston arrangement. Such devices are commercially available.

Alternatively, a squeeze bottle with an aperture or opening dimensioned to aerosolize an aerosol formulation by forming a spray when squeezed can be used. The opening is usually found in the top of the bottle, and the top is generally tapered to partially fit in the nasal passages for efficient administration of the aerosol formulation. Preferably, the nasal inhaler can provide a metered amount of the aerosol formulation, for administration of a measured dose of the therapeutic agent.

In some embodiments, the aerosolization of a liquid formulation for inhalation into the lung involves a propellant. The propellant may be any propellant generally used in the art. Specific non-limiting examples of such useful propellants are a chlorofluorocarbon, a hydrofluorocarbon, a hydrochlorofluorocarbon, or a hydrocarbon, including trifluoromethane, dichlorodifluoromethane, dichlorotetrafluoroethanol, and 1,1,1,2-tetrafluoroethane, or combinations thereof.

The present disclosure also provides aerosol formulations and dosage forms for use in treating subjects with various lung diseases. In general, such dosage forms contain the therapeutic agent in a pharmaceutically acceptable diluent. Pharmaceutically acceptable diluents in such aerosol formulations include but are not limited to sterile water, saline, buffered saline, dextrose solution, and the like. In certain embodiments, a diluent that may be used in the present invention or the pharmaceutical formulation is phosphate buffered saline or a buffered saline solution generally between the pH 7.0-8.0 range (e.g., pH 7.4), or water.

The aerosol formulation also may optionally include pharmaceutically acceptable carriers, diluents, solubilizing or emulsifying agents, surfactants and excipients.

The formulation can include a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable plasma half-life for clearance. Such macromolecules include but are not limited to soy lecithin, oleic acid and sorbitan trioleate.

The formulations can also include other agents useful for pH maintenance, solution stabilization, or for the regulation of osmotic pressure. Examples of the agents include but are not limited to salts, such as sodium chloride, or potassium chloride, and carbohydrates, such as glucose, galactose or mannose, and the like.

The present disclosure further contemplates aerosol formulations comprising the composition as described herein and another therapeutically effective agent.

In some embodiments, the composition can be administered into blood vessels. The blood can carry the mitochondria or the combined mitochondria agent to a target site, for example, an organ, a tissue, or an injured site. For example, pulmonary blood vessels (e.g., pulmonary veins) can carry the mitochondria or the combined mitochondrial agent to the lung. In some cases, the composition described herein can be used to improve the function of the lung, ears, eyes, and skins.

In some embodiments, the compositions are administered by the oral or nasal respiratory route for local or systemic effect. Compositions in pharmaceutically acceptable solvents can be nebulized by use of inert gases. Nebulized solutions can be inhaled directly from the nebulizing device or the nebulizing device can be attached to a face mask, or intermittent positive pressure breathing machine. Solution, suspension, or powder compositions can be administered, orally or nasally, from devices that deliver the formulation in an appropriate manner. For administration by inhalation, the compositions can be delivered in the form of an aerosol spray from a pressured container or dispenser that contains a suitable propellant, e.g., a gas such as carbon dioxide, or a nebulizer. Such methods include those described in U.S. Pat. Nos. 6,468,798; 6,695,227; 7,431,222; US20050224608; each of which is incorporated herein by reference in its entirety.

Thus, in some embodiments, the aerosol composition can be administered to respiratory track, lungs, ears, eyes, nasal passage, rectum, skins, and head. In some embodiments, the composition is delivered to lungs, nasal passage, rectum, intestine, skin, and eyes to treat cancers or tumors in these sites.

In some embodiments, the composition is administered through a metered dose sprayer, a squeeze bottle, a pressurized metered dose inhaler, a ventilator, or face mask and tent. Systems of aerosol delivery, such as the pressurized metered dose inhaler and the dry powder inhaler are disclosed in Newman, S. P., Aerosols and the Lung, Clarke, S. W. and Davia, D. editors, pp. 197-22, which is incorporated by reference herein in its entirety. The subject can also inhale the composition through a ventilator, which will be set to a flow rate based on patient comfort and needs. This is determined by pulmonary graphics (i.e., respiratory rate, tidal volumes, etc.). The composition can also be prepared to allow inhalation by the subject using a facemask or tent. In certain cases, the composition can be packaged into a portable device (e.g., a portable inhaler device) and administered in a metered dose, for example, to permit intermittent treatment of a recipient who is not in a hospital setting. Different concentrations of composition can be packaged in the containers.

A convenient way to effectively deliver the composition to the alveoli is to select a nebulizer which produces sufficiently small particles (e.g., producing particles with a mean particle diameter of less than 5.0 microns (μm), more preferably having a mean particle diameter of about 0.2 to about 4.0 μm, and most preferably having a mean diameter of about 0.2 to about 2 μm). As an alternative to selecting small mean particle diameters to achieve substantial alveoli deposition, a very high dosage of the composition can be administered, with a larger mean particle diameter. In such an approach, it is beneficial if the particular composition is not too irritating at the required dosage and that there are a sufficient number of particles in the total population having a diameter in the 0.5 to about 5 μm range to allow for deposition in the alveoli. For proximal airway delivery, the mean particle size may be larger. For example, suitable mean particle diameters may generally be less than about 15 μm, e.g., from about 4 μm to about 15 μm, e.g., from about 5 μm to about 10 μm. The composition can be aerosolized by any appropriate method. For use with humans or other primates, the aerosol can be generated using a medical nebulizer system which delivers the aerosol through a mouthpiece, facemask, etc., from which the subject can draw the aerosol into the lungs. Various nebulizers are known in the art and can be used in the method as described herein. See, e.g., U.S. Pat. Nos. 4,268,460; 4,253,468; 4,046,146; 3,826,255; 4,649,911; 4,510,829; each of which is incorporated herein by reference in its entirety. The selection of a nebulizer system will depend on whether alveolar or airway delivery (i.e., trachea, primary, secondary or tertiary bronchi, etc.) is desired.

Examples of nebulizers useful for alveolar delivery include the Acorn 1 nebulizer, and the Respirgard II™ Nebulizer System, both available commercially from Marquest Medical Products, Inc., Inglewood, Colo. Other commercially available nebulizers for use with the instant invention include the UltraVent™ nebulizer available from Mallinckrodt, Inc. (Maryland Heights, Mo.); the Wright nebulizer (Wright, B. M., Lancet (1958) 3:24-25); and the DeVilbiss nebulizer (Mercer et al., Am. Ind. HYR. Assoc. J. (1968) 29:66-78; T. T. Mercer, Chest (1981) 80:6 (Sup) 813-817). Nebulizers useful for airway delivery include those typically used in the treatment of asthma. Such nebulizers are also commercially available. One of skill in the art can determine the usefulness of a particular nebulizer by measuring the mean particle size generated thereby with e.g. a 7 stage Mercer cascade impactor (Intox Products, Albuquerque, N. Mex.).

The total amount of the composition delivered to the subject will depend upon several factors, including the total amount aerosolized, the type of nebulizer, the particle size, subject breathing patterns, severity of lung disease, and concentration in the aerosolized solution, and length of inhalation therapy. The amount of composition measured in the alveoli may be substantially less than what would be expected to be from the amount of composition present in the aerosol, since a large portion of the composition may be exhaled by the subject or trapped on the interior surfaces of the nebulizer apparatus.

Skilled practitioners will be able to readily design effective protocols, particularly if the particle size of the aerosol is optimized. In some instances, it is useful to administer higher doses when treating more severe conditions. If necessary, the treatment can be repeated on an adhoc basis depending upon the results achieved. If the treatment is repeated, the mammalian host can be monitored to ensure that there is no adverse immune response to the treatment. The frequency of treatments depends upon a number of factors, such as the amount of composition administered per dose, as well as the health and history of the subject. As used herein, the “amount nebulized” or “amount aerosolized” of the composition means the amount that actually leaves the apparatus as an aerosol, i.e., the amount placed into the apparatus less the amount retained in the reservoir and on the inner surfaces of the apparatus at the conclusion of a treatment session.

### Lung Disorders

The therapeutic agents described herein can be used to treat various lung disorders. The subject can have any lung disease that may benefit from the treatment, e.g., ischemia, perfusion injury, pulmonary hypoplasia, congenital diaphragmatic hernia (CDH), Poland syndrome, Chest wall diseases (e.g., pectus excavatum), pneumonectomy, bronchopulmonary dysplasia, lung injury, and inhalation injury, asthma, cystic fibrosis, etc. For example, the subject may have pulmonary hypoplasia. In some embodiments, an effective amount of composition comprising isolated mitochondria can be administered to the subject, for example, through respiratory tract.

In some embodiments, the methods described herein can be used to treat acute respiratory distress syndrome (ARDS), acute lung injury (ALI), respiratory failure, reduced respiratory function, and/or lung inflammation.

In some embodiments, the methods described herein can be used to treat pulmonary fibrosis, lung infection (e.g., by virus, coronavirus, flu, bacteria, or fungi), asthma, chronic obstructive pulmonary disease, pulmonary emboli, lung cancer, pulmonary hypertension, or lymphangioleiomyomatosis. In some embodiments, the methods described herein can be used to treat pneumonia.

In some embodiments, the subject may be identified as having insufficient, incomplete, or slow lung development. Alternatively or in addition, the subject may have some damage in the lungs. For example, the subject can be a habitual smoker or a worker frequently exposed to smoke inhalation.

### Treating Ischemia-Related Injuries

Methods described herein can be used to treat ischemic lungs. For example, an effective amount of isolated mitochondria can be administered through the respiratory tract. The mitochondria can be internalized by the cells in the lungs.

Reperfusion injury is the tissue damage by blood supply when blood returns to the tissue after a period of ischemia or lack of oxygen. The absence of oxygen and nutrients during the ischemic period results in inflammation and oxidative damage when blood flow is restored. The inflammatory response further leads to the reperfusion injury in the tissue. Therefore, in some instances, a treatment also involves administering immune suppressors to the patient. The immune suppressors can be e.g., administrated separately, but as a concurrent treatment with the mitochondrial agent. Alternatively, or in addition, the immune suppressors can be linked to mitochondria to form a combined mitochondrial agent, which can be used for the treatment. Particularly useful immune suppressors are bisphosphonates.

In some embodiments, the subject has acute lung injury (ALI). Acute lung injury is often associated with acute respiratory failure and refractory hypoxemia. In some embodiments, the subject has bilateral pulmonary infiltrates that are consistent with pulmonary edema in the absence of left atrial hypertension. ALI complicates a wide variety of medical and surgical conditions and reflects the pulmonary response to lung insult or precipitating factors. The most common reason for ALI is ischemia/reperfusion injury (IRI). IRI can occur with extracorporeal gas exchange, cardiopulmonary bypass and lung ventilation and has been shown to have profound effects on lung function and cellular viability and to significantly contribute to increased morbidity and mortality in both pediatric and adult patients. The incidence of ALI in adult patients undergoing cardiac surgery has been estimated to range from 53.8% to 73.5%. In pediatric patients, ALI is present in 8-10% of patients requiring mechanical ventilation. In both pediatric and adult patients, ALI has an estimated mortality of 20-75%. At present, there are no viable methodologies to treat ALI occurring through IRI with a goal to increase lung survival and lung function.

Methods described herein can be used to treat ALI. For example, an effective amount of aerosolized composition comprising mitochondria or combined mitochondrial agents can be administered to the respiratory tract of a subject. For example, about or at least 1×10⁷ of mitochondria can be administered into the lung. The injected mitochondria are internalized by endothelial cells and can provide enhanced oxygen consumption, reduce lung resistance and elastance, increase compliance, and/or reduce ischemic area.

### Vasodilation and Blood Flow

The methods described herein can significantly increase blood flow without altering mean blood pressure or heart rate. The ability to increase blood flow with no increase in heart rate allows for clinical usage in angina type injury and in ischemia/reperfusion related injury and in tissue damage areas where increased blood flow and oxygen delivery would be needed. Thus, the methods described herein can be used in artery interventions to remove clots or obstructions in blood vessels.

Methods described herein can also be used to increase blood flow and/or oxygen delivery for various organs or tissues (e.g., heart, or lungs), and induce vascular dilatation in the organs. In some instances, the isolated mitochondria or combined mitochondrial agents can be used to decrease vascular resistance in an organ (e.g., heart, or lung). Isolated mitochondria or combined mitochondrial agents can be used to increased blood flow. The isolated mitochondria and/or combined mitochondrial agents can be added to a contrast agent, and can be used in the identification and removal of blockages in the organs.

It is noted that the effects of the compositions are dependent on time from isolation to time of use. The vasodilatory effects decreases as time from isolation is extended. While not intending to be bound by any theory, it is hypothesized that freshly isolated mitochondria have certain chemicals, which can increase blood flow. Therefore, in some methods, the mitochondria are freshly isolated and viable. For example, the mitochondria or combined mitochondrial agents are administered to a subject within about 5 minutes, about 10 minutes, about 20 minutes, about 30 minutes, about 40 minutes, about 50 minutes, about 60 minutes, about 70 minutes, about 80 minutes, about 90 minutes, about 100 minutes, about 110 minutes, about 120 minutes after the time point when the mitochondria isolation process starts or after the mitochondria are isolated. In some cases, the mitochondria or combined mitochondrial agents are administered to a subject within about 20 minutes to about 60 minutes (e.g., about 20 minutes, about 30 minutes, about 40 minutes, about 50 minutes, about 60 minutes) after the time point when the mitochondria isolation process starts or after the mitochondria are isolated.

In some cases, increasing blood flow is not desirable (e.g., treating ischemia/reperfusion in lungs, treating cancer). In these cases, mitochondria or combined mitochondrial agents can be stored for a short period of time (e.g., from about 30 to about 60 minutes) before usage. This method can be used to increase tissue viability (e.g., treating ischemia/reperfusion injury) without causing an increase in blood flow. In these cases, the mitochondria or combined mitochondrial agents are administered to a subject at least 60 about minutes (e.g., about 65 minutes, about 70 minutes, about 80 minutes, about 90 minutes, about 100 minutes, about 110 minutes, about 120 minutes) after the time point when the mitochondria isolation process starts or after the mitochondria are isolated.

### Imaging

Imaging agents can be attached to mitochondria, often by co-incubation of the mitochondria with the imaging agents. Such imaging agents include, but are not limited to, MitoTracker and pHrodo fluorophores from Thermo Fisher Scientific Inc., ¹⁸F-Rhodamine 6G, and iron oxide nanoparticles.

Combined mitochondrial agents that include an imaging agent can be administered to the subject through respiratory tract. Tissues containing the labeled mitochondria can be examined using imaging techniques, such as positron emission tomography (PET), microcomputed tomography (μCT), and magnetic resonance imaging (MRI), brightfield microscope, and 3-D super-resolution microscopy, etc. Skilled practitioners will appreciate that other imaging techniques or modalities may be used. They include, but are not limited to, x-rays, scintigraphy, fluorescence and ultrasound.

Positron emission tomography is an imaging technique that produces a three-dimensional image in the body, and can be used in methods described herein. The system detects pairs of gamma rays emitted indirectly by a positron-emitting radioisotope (tracer). Three-dimensional images of tracer concentration within the body are then constructed by computer analysis. Useful reporter groups include radioactive isotopes, such as ¹¹C, ¹³N, ¹⁵O, ¹⁸F, ⁶⁴Cu, ⁶⁸Ga, ⁸¹mKr, ⁸²Rb, ⁸⁶Y, ⁸⁹Zr, ¹¹¹In, ¹²³I, ¹²⁴I, ¹³³Xe, ²⁰¹Tl, ¹²⁵I, ³⁵S, ¹⁴C, ³H. In some methods, mitochondria can be labeled by radioactive isotopes, e.g., ¹⁸F, or by molecules that incorporate radioactive isotopes, e.g., ¹⁸F-Rhodamine 6G, ¹⁸F-labeled rhodamine B. After the mitochondria are internalized by target cells, the PET imaging technique, or similar technique, can be employed to view the target cells.

Magnetic resonance imaging is a medical imaging technique to image the anatomy and the physiological processes of the body and can be used in methods described herein. In some instances, it can be used in conjugation with some other imaging techniques, for example, PET. Images acquired from both devices can be taken sequentially, in the same session, and combined into a single superposed (co-registered) image. PET/MRI scans can be used to diagnose a health condition in humans and animals, e.g., for research, medical, and agricultural purposes.

Micro-computed tomography uses x-rays to create cross-sections of a physical object that can be used to recreate a virtual model without destroying the original object, and can be used in methods described herein. In some instances, it is used in conjugation with some other imaging techniques, for example, PET. Images acquired from both devices can be taken sequentially, in the same session, and combined into a single superposed (co-registered) image. Thus, functional imaging obtained by PET, which depicts the spatial distribution of metabolic or biochemical activity in the body can be more precisely aligned or correlated with anatomic imaging obtained by CT scanning. Two- and three-dimensional image reconstruction may be rendered as a function of a common software and control system.

3D-structured illumination microscopy, 3D-SIM, or 3-D super-resolution microscopy, allows complete 3D visualization of structures inside cells and can be used in the methods described herein. Structured illumination microscopy is an imaging method capable of doubling the spatial resolution of conventional widefield fluorescence microscopy by using spatially structured illumination light. It enhances spatial resolution by collecting information from frequency space outside the observable region.

The described methods, i.e., methods that include administering mitochondria and/or combined mitochondrial agents, are useful for diagnosing a variety of diseases, such as cancers, (e.g., lung, brain, pancreatic, melanoma, prostate, colon cancers), cardiovascular disease (e.g., myocardial infarction, atherosclerosis), autoimmune diseases (e.g., multiple sclerosis, diabetes, irritable bowel syndrome, Celiac disease, Crohn's disease), and inflammatory disease.

Methods using agents for imaging purpose are well-known in the art and described in, for example, Bartholomä et al., Biological characterization of F18-labeled Rhodamine B, a potential positron emission tomography perfusion tracer, Nucl Med Biol 40, 1043-1048, PMC3820364 (2013); Bartholomä et al., ¹⁸F-labeled rhodamines as potential myocardial perfusion agents: comparison of pharmacokinetic properties of several rhodamines, Nucl Med Biol 42, 796-803, PMC4567415 (2015); and Pacak et al., Superparamagnetic iron oxide nanoparticles function as a long-term, multi-modal imaging label for non-invasive tracking of implanted progenitor cells, PLoS ONE 9, e108695, PMC4177390 (2014). Each of the foregoing can be useful in methods described herein and is incorporated herein by reference its entirety.

### Drug Delivery

The present specification provides methods to deliver pharmaceutic agents, e.g., to cells and/or tissues of a patient. Mitochondria are taken up by tissue cells through an actin-dependent internalization process, thereby providing a way to deliver pharmaceutic agents directly into the cells. In some instances, combined mitochondrial agents enter into lung tissues or blood vessels through the respiratory tract.

An antibody or an antigen-binding fragment can be linked or attached to mitochondria. Skilled practitioners will appreciate that linking the antibody or antigen binding fragment to mitochondria or combined mitochondrial agent can allow the mitochondria or combined mitochondrial agent to be targeted to specific sites, e.g., to target cells and/or tissues. In some instances, the antibody or the antigen-binding fragment are designed to target specific cell types, for example, smooth muscle cells in lung, immune cells, macrophages, etc.

In some embodiments, the present disclosure provides methods of delivering a nucleic acid (e.g., DNA, RNA, miRNA), a vector, a peptide, or a protein to a target site (e.g., lung tissue). Isolated mitochondria can be used as a carrier to deliver a nucleic acid or a peptide into a cell. In some instances, combined mitochondrial agents that include nucleic acid polymers can be administered to a subject to replace a mutated gene in the subject that causes disease, to inactivate, or “knock out,” a mutated gene, or to introduce a new gene into the subject. Exemplary nucleic acid polymers include, but are not limited to, double-stranded DNA, single-stranded DNA, double-stranded RNA, single-stranded RNA, or triple helix nucleic acid molecules. In certain instances, the nucleic acid polymers are DNA, interfering RNAs (siRNA), and micro RNAs.

### Minimizing Cardiotoxicity

Chemotherapy is a common treatment for various cancers, however, it also causes several serious complications. Chemotherapy-induced cardiotoxicity is one complication that limits the clinical use of chemotherapeutic agents. Certain chemotherapeutic agents, such as anthracyclines, are highly effective against acute lymphoblastic and myeloblastic leukemias, but are particularly harmful to the heart due to its effects on mitochondria. The damage to mitochondria further leads to chemotherapy-induced cardiotoxicity. Angsutararux et al., Chemotherapy-Induced Cardiotoxicity: Overview of the Roles of Oxidative Stress. Oxid Med Cell Longev. 2015; 2015:795602. doi: 10.1155/2015/795602 (2015); Guo et al., Cardiovascular toxicities from systemic breast cancer therapy, Front Oncol. 4:346. doi: 10.3389/fonc.2014.00346. eCollection (2014).

The present disclosure provides methods to minimize chemotherapy-induced cardiotoxicity. The methods involve administering an effective amount of isolated mitochondria and/or a combined mitochondrial agent to a patient through respiratory tract. If the patient needs to be treated with chemotherapy (e.g., because prescribed by a physician or veterinarian), the patient can be treated with mitochondria and/or combined mitochondrial agent, before, during, and/or after administration of the chemotherapy. For example, patients can be treated with mitochondria and/or combined mitochondrial agent starting immediately after administration, as a singular treatment or continuing intermittently or continuously for about 1, 2, 5, 8, 10, 20, 30, 50, or 60 days, one year, indefinitely, or until a physician determines that administration of the mitochondria and/or combined mitochondrial agent is no longer necessary.

### Organ/Tissue Transplantation

The present disclosure also features methods of transplanting an organ(s), tissues, masses of cells and/or isolated cells. The methods can include a step of exposing the organ(s), tissues, mass of cells and/or isolated cells to mitochondria or combined mitochondrial agents prior to transplantation. Such exposures can occur in situ and/or ex vivo. The organ(s), tissues and/or isolated cells may be exposed to a composition comprising mitochondria or combined mitochondrial agents (e.g., an aerosolized composition).

Exposure of an organ or tissue to compositions comprising mitochondria or combined mitochondrial agents can be performed ex vivo and/or in situ by any method known in the art. For example, the exposure may be performed ex vivo in any chamber or space having sufficient volume for submerging the organ or tissue, completely or partially, in the composition. As another example, the organ may be exposed to compositions comprising mitochondria or combined mitochondrial agents by placing the organ in any suitable container, and causing the compositions comprising mitochondria or combined mitochondrial agents to “wash over” the organ, such that the organ is exposed to a continuous flow of the composition.

An effective amount of mitochondria or combined mitochondria agents is an amount that is effective for enhancing survival and/or improving function of organs, or cells in vivo and/or in vitro. Within the context of transplantation of individual cells or masses of cells, e.g., transplant donors and/or recipients, an effective amount of mitochondria or combined mitochondria agents is an amount that is administered to the transplant donor and/or recipient sufficient to enhance survival of the cell or mass of cells, e.g. to reduce loss of the cell, or mass of cells, and/or to improve functional performance of a transplanted cell or a mass of cells. Within the context of transplantation of organs and tissues, e.g., transplant donors and/or recipients, an effective amount of mitochondria or combined mitochondria agents is an amount that is administered to the transplant donor and/or recipient sufficient to enhance survival of the organ, tissue or cells of interest, e.g., to reduce loss of cells from which the organ or tissue is composed, and/or to improve functional performance of an organ.

In some instances, the administration of the composition through a respiratory tract is performed before the organ is retrieved from the donor. In some instances, the administration of the composition through a respiratory tract is performed after the organ is transplanted into the recipient. In some instances, administration of the composition (e.g., aerosolized composition) are performed before organ retrieval, after harvesting of the organ, and then again after implantation into the recipient. In some instances, the administration of the composition is performed during the transplantation surgery (e.g., lung transplantation, heart transplantation).

### Treatment of Cancer

Methods described herein also provide treatment of cancers (e.g., lung cancers). After the compositions as described herein are administered to the subject through respiratory tract, the mitochondria or the combined mitochondrial agents can be taken up by the tumor cells. In some embodiments, a cytostatic agent or cytotoxic agent can be delivered to the tumor to kill cancer cells. In some embodiments, the therapeutic agent is a chemotherapeutic agent, for example, anthracycline.

Cancers that may be treated using the methods and compositions of the present disclosure include, e.g., mouth/pharynx cancer, esophagus cancer, larynx cancer, lung cancer, or any cancer in the respiratory tract.

Further, in some embodiments, an antibody or an antigen-binding fragment can be linked or attached to mitochondria. Skilled practitioners will appreciate that linking the antibody or antigen binding fragment to mitochondria or combined mitochondrial agents can allow the mitochondria or combined mitochondrial agents to target specific sites, e.g., to target cells and/or tissues. In some instances, the antibody or the antigen-binding fragment are designed to target specific cell types, for example, cancer cells.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic agent (i.e., an effective dosage) depends on the therapeutic agents selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic agents described herein can include a single treatment or a series of treatments.

Dosage, toxicity and therapeutic efficacy of the therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Agents which exhibit high therapeutic indices are preferred. While agents that exhibit toxic side effects may be used, care should be taken to design a delivery system that targets such agents to the site of affected tissue in order to minimize potential damage to uninfected cells and, thereby, reduce side effects. How to estimate a safe range for a human patient is described, e.g., in Food and Drug Administration. “Guidance for industry: estimating the maximum safe starting dose in initial clinical trials for therapeutics in adult healthy volunteers.” Center for Drug Evaluation and Research (CDER) (2005), which is incorporated by reference herein in its entirety.

The data obtained from cell culture assays and animal studies can be used in formulating a range of dosage for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. Such information can be used to more accurately determine useful doses in humans. Levels in plasma may be measured, for example, by high performance liquid chromatography.

Skilled practitioners will appreciate that the amount of mitochondria and/or combined mitochondrial agents, e.g., compositions comprising mitochondria and/or combined mitochondrial agents, that should be administered to a patient will vary depending upon, e.g., the type of disorder being treated, the route of administration, the duration of the treatment, the size of an area to be treated, and/or the location of the treatment site in the patent, among others. Skilled practitioners will be able to determine dosages to be administered depending on these and other variables. For example, a total of about 1×10¹⁰ to 1×10¹⁴ of mitochondria can be administered to a subject (e.g., to treat localized ischemia in the lungs). In the case of small focal lesions, 1×10³ to 1×10⁶ mitochondria can be administered to the patient. Therefore, an effective amount of mitochondria or combined mitochondrial agents (or compositions comprising same) is the total amount of mitochondria or combined mitochondrial agents sufficient to bring about a desired therapeutic effect. An effective amount can be, e.g., at least or about 1×10² mitochondria or combined mitochondrial agents e.g., from about 1×10³ to about 1×10¹⁴, about 1×10⁴ to about 1×10¹³, about 1×10⁵ to about 1×10¹², about 1×10⁶ to about 1×10¹¹, about 1×10⁷ to about 1×10¹⁰, about 1×10³ to about 1×10⁷, about 1×10⁴ to about 1×10⁶, about 1×10⁷ to about 1×10¹⁴, or about 1×10⁸ to about 1×10¹³, about 1×10⁹ to about 1×10¹², about 1×10⁵ to about 1×10⁸; or at least or about 1×10³, 1×10⁴, 1×10⁵, 1×10⁶, 1×10⁷, 1×10⁸, 1×10⁹, 1×10¹⁰, 1×10¹¹, 1×10¹², 1×10¹³, or at least or about 1×10¹⁴, or e.g., an amount more than 1×10¹⁴. As used herein, the term “total amount” in the context of administration to a patient can refer to the total amount of mitochondria or combined mitochondrial agents in a single administration or in multiple administrations, depending on the dosing regimen being performed.

### Pharmaceutical Compositions

The disclosure provides compositions that comprise isolated mitochondria, compositions that comprise combined mitochondrial agents, compositions that comprise both isolated mitochondria and combined mitochondrial agents, and methods of using such compositions.

A pharmaceutical composition described herein may include mitochondria and/or combined mitochondria agents and a pharmaceutically acceptable carrier. As used herein, the language “pharmaceutically acceptable carrier” includes saline, solvents, dispersion media, coatings, antibacterial and antifungal agents, isotonic and absorption delaying agents, and the like, compatible with pharmaceutical administration. In some embodiments, the pharmaceutically acceptable carrier is phosphate buffered saline, saline, Krebs buffer, Tyrode's solution, contrast media, or omnipaque, or a mixture thereof. In some embodiments, the pharmaceutically acceptable carrier is sterile mitochondria buffer (300 mM sucrose; 10 mM K+-HEPES (potassium buffered (4-(2-hydroxyethyl)-1-piperazineethanesulfonic acid, pH 7.2); 1 mM K+-EGTA, (potassium buffered ethylene glycol tetraacetic acid, pH 8.0)). In some embodiments, the pharmaceutically acceptable carrier is respiration buffer (250 mM sucrose, 2 mM KH₂PO₄, 10 mM MgCl₂, 20 mM K-HEPES Buffer (pH 7.2), and 0.5 mM K-EGTA (pH 8.0)).

Pharmaceutical compositions are typically formulated to be compatible with its intended route of administration.

A pharmaceutical composition can be formulated for various clinical uses, e.g., imaging, treating wounds, treating injuries, preserving organs, improving mitochondrial functions in organs or tissues, and skin care. In some cases, the pharmaceutically acceptable carrier is a contrast agent for imaging purpose. In some embodiments, the pharmaceutical composition may include antiseptic agents, antibacterial agents (e.g., antibiotics), antifungal agents, disinfectants, analgesic agents, anesthetic agents, steroids, nutritional supplements, ethereal oils, etc. An anesthetic agent is a drug that can prevent pain during surgery or treatment. Exemplary analgesic agents include, without limitation, paracetamol, nonsteroid anti-inflammatory drugs, salicylates, ibuprofen and lidocaine. Exemplary antibacterial agents include, without limitation, dichlorobenzyl alcohol, amylmetacresol and antibiotics. Exemplary antibiotics include penicillins carbapenems, cephalosporins aminoglycosides, bacitracin, gramicidin, mupirocin, chloramphenicol, thiamphenicol, lincomycin, clindamycin, macrolides, novobiocin, polymyxins, rifamycins, spectinomycin, tetracyclines, vancomycin, teicoplanin, streptogramins, anti-folate agents, sulfonamides, trimethoprim, pyrimethamine, nitrofurans, methenamine mandelate, methenamine hippurate, nitroimidazoles, quinolones, fluoroquinolones, isoniazid, ethambutol, pyrazinamide, para-aminosalicylic acid, cycloserine, capreomycin, ethionamide, prothionamide, thiacetazone and viomycin. Antiseptic agents are antimicrobial substances that can be applied to living tissue/skin to reduce the possibility of infection, sepsis, or putrefaction. Exemplary antiseptics include, without limitation, chlorhexidine and salts thereof, benzalkonium and salts thereof, triclosan and cetylpyridium chloride. Exemplary antifungal agents include, without limitation, tolnaftate, miconazole, fluconazole, clotrimazole, econazole, ketoconazole, itraconazole, terbinafine, amphotericin, nystatin and natamycin. Exemplary steroids include, without limitation, prednisone acetate, prednisone valerate, prednisolone, alclometasone dipropionate, fluocinolone acetonide, dexamethasone, methylprednisolone, desonide, pivolate, clocortolone pivolate, triamcinolone acetonide, prednicarbate, fluticasone propionate, flurandrenolide, mometasone furoate, desoximetasone, betamethasone, betamethasone dipropionate, betamethasone valerate, betamethasone propionate, betamethasone benzoate, diflorasone diacetate, fluocinonide, halcinonide, amcinonide, halobetasol propionate, and clobetasol propionate. Exemplary nutritional supplements include, without limitation, vitamins, minerals, herbal products and amino acids. Vitamins include without limitation, vitamin A, those in the vitamin B family, vitamin C, those in the vitamin D family, vitamin E and vitamin K. Ethereal oils include without limitation, those derived from mint, sage, fir, lavender, basil, lemon, juniper, rosemary, eucalyptus, marigold, chamomile, orange and the like. Many of these agents are described, e.g., in WO 2008152626, which is incorporated by reference in its entirety.

Compositions comprising mitochondria and/or combined mitochondrial agents can be formulated in any form, e.g., liquids, semi-solids, or solids. Exemplary compositions include liquids, creams, ointments, salves, oils, emulsions, liposome formulations, among others.

Isolated mitochondria or combined mitochondrial agents can be included in compositions that are designed for use in organ, tissue, or cell transplantation. The composition may include isolated mitochondria and/or combined mitochondrial agents and a liquid that is suitable for administration to patients and/or organs in situ or ex vivo, e.g., for maintaining organs, tissues or cells ex vivo. In general, the liquid will be an aqueous solution. Examples of solutions include Phosphate Buffered Saline (PBS), Celsior™ solution, Perfadex™ solution, Collins solution, citrate solution, tissue culture media (e.g., Dulbecco's Modified Eagle's Medium (DMEM)), the Histidine-tryptophan-ketoglutarate (HTK) solution, and the University of Wisconsin (UW) solution (Oxford Textbook of Surgery, Morris and Malt, Eds., Oxford University Press, 1994).

The University of Wisconsin cold storage solution is considered a standard solution for organ transplantation. It includes the following: 100 mM potassium lactobionate, 25 mM KH₂PO₄, 5 mM MgSO₄, 30 mM raffinose, 5 mM adenosine, 3 mM glutathione, 1 mM allopurinol, and 50 g/L hydroxyethyl starch. Isolated mitochondria or combined mitochondrial agents can be added to these liquids for organ, tissue and cell preservation.

When administered through certain routes, such as the respiratory tract (e.g., nose, a tracheostomy, or by endotracheal tube (ETT)), surfactants can allow the composition to reach the distal airways. Thus, in some embodiments, compositions described herein (e.g., liquid solutions, aqueous solutions (where water-soluble), aerosol formulations, dispersions, sterile powders etc.) can comprise one or more surfactants.

The surfactant can be selected from the group consisting of non-ionic, cationic, anionic, and zwitterionic surfactants. Mixtures of surfactants can also be used. Exemplary classes of surfactants include alcohol ether sulfates, alcohol sulfates, alkanolamides, alkyl sulfonates, amine oxides, amphoteric surfactants, anionic surfactants, betaine derivatives, cationic surfactants, disulfonates, dodecylbenzene, sulfonic acid, ethoxylated alcohols, ethoxylated alkyl phenols, ethoxylated fatty acids, glycerol esters hydrotropes, lauryl sulfates, mono and diglycerides, non-ionic surfactants, phosphate esters, quaternary surfactants, and sorbitan derivatives.

To be suitable for delivery through respiratory tract, some other appropriate pharmaceutical carriers can also be used. These carries include e.g., liposomes, nano- and microparticles, cyclodextrins, microemulsions, micelles, suspensions, or solutions. In some embodiments, these carriers are made of lipids (e.g., liposomes, niosomes, microemulsions, lipidic micelles, solid lipid nanoparticles) or composed of polymers (e.g., polymer micelles, dendrimers, polymeric nanoparticles, nonogels, nanocapsules). These carriers can carry the therapeutic agent to the lung in a controlled manner. Nanocarriers can also be used. Localized therapy of the target organ generally requires smaller total doses to achieve clinically effective results. To reach this goal, nanocarriers can be engineered to slowly degrade, react to stimuli and to be site specific.

Pharmaceutical compositions described herein can be included in a container, pack, or dispenser together with instructions for administration. Pharmaceutical compositions described herein can also be prepared in a form that is suitable to be converted to an aerosolized form (e.g., by an appropriate nebulizer).

### EXAMPLES

The invention is further described in the following examples, which do not limit the scope of the invention described in the claims.

### Example 1: Isolating Mitochondria from Autologous Skeletal Muscle from Subject

Mitochondria were isolated from autologous skeletal muscle tissue. The source of tissue was dependent upon the surgical entry point and access. Highly efficacious mitochondrial transplantation typically requires freshly isolated, viable, and functional mitochondria. Non-viable mitochondria (e.g., previously frozen mitochondria), mitochondrial fractions (proteins, complex I-V), mitochondrial DNA and RNA, and exogenous ATP or ADP usually do not have sufficient functionality.

### Methods

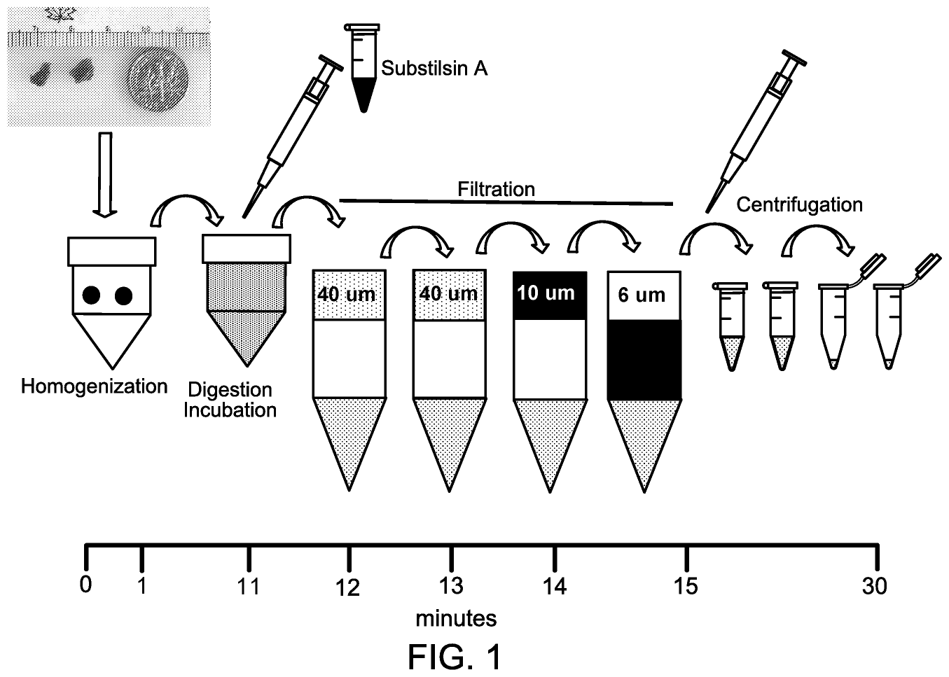

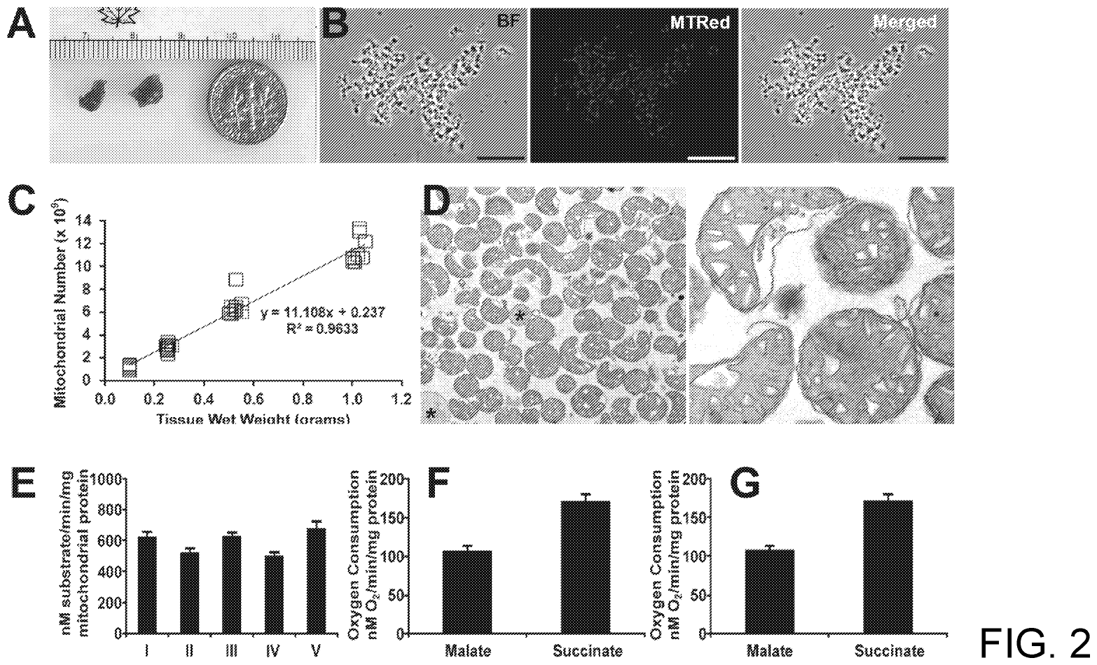

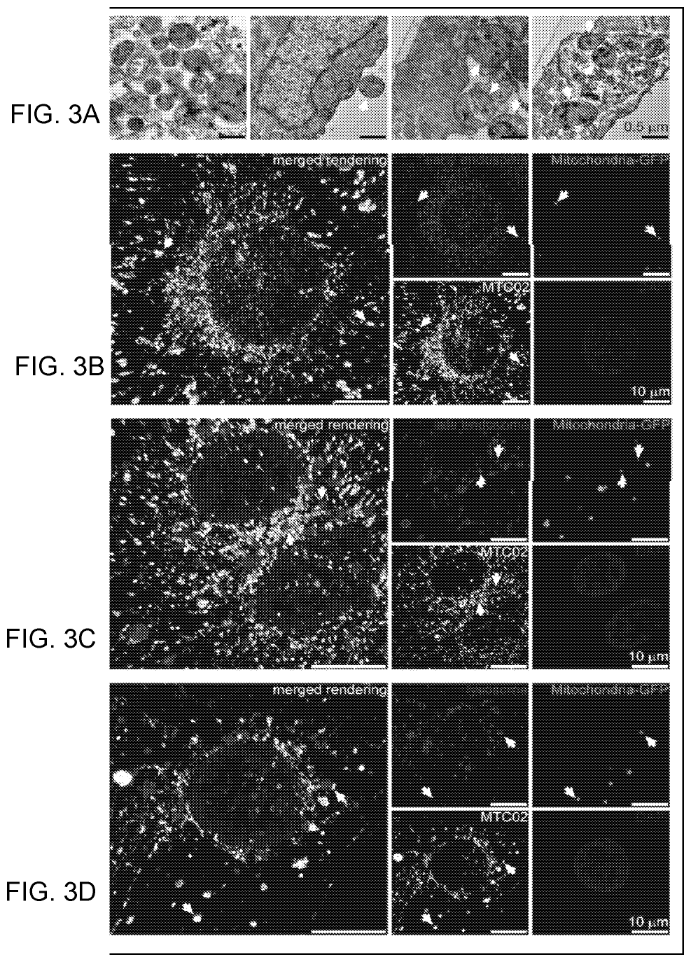

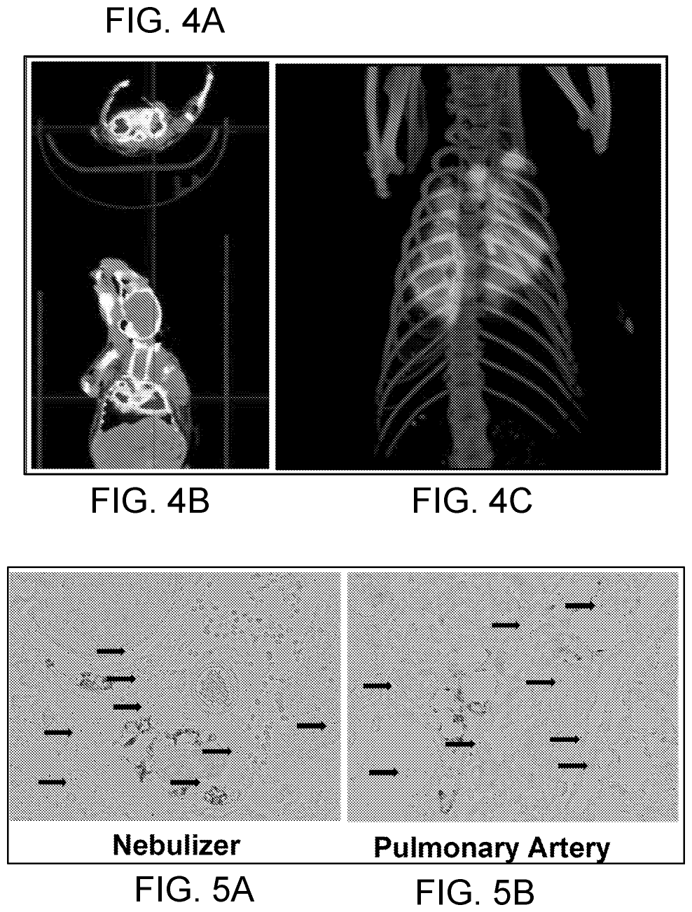

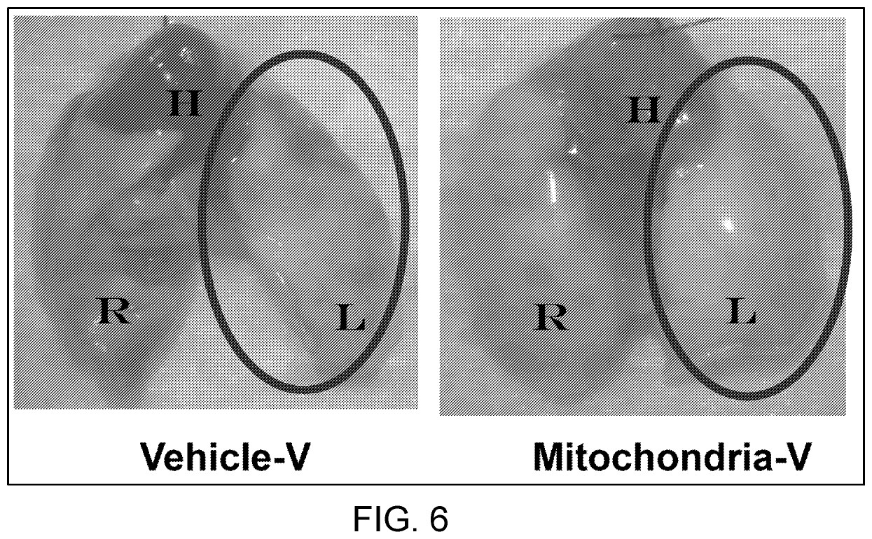

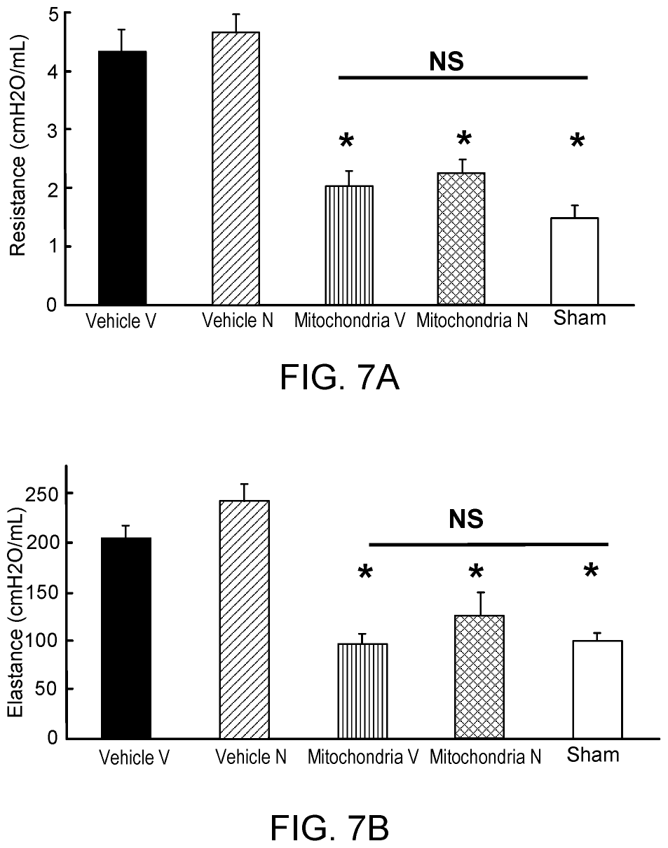

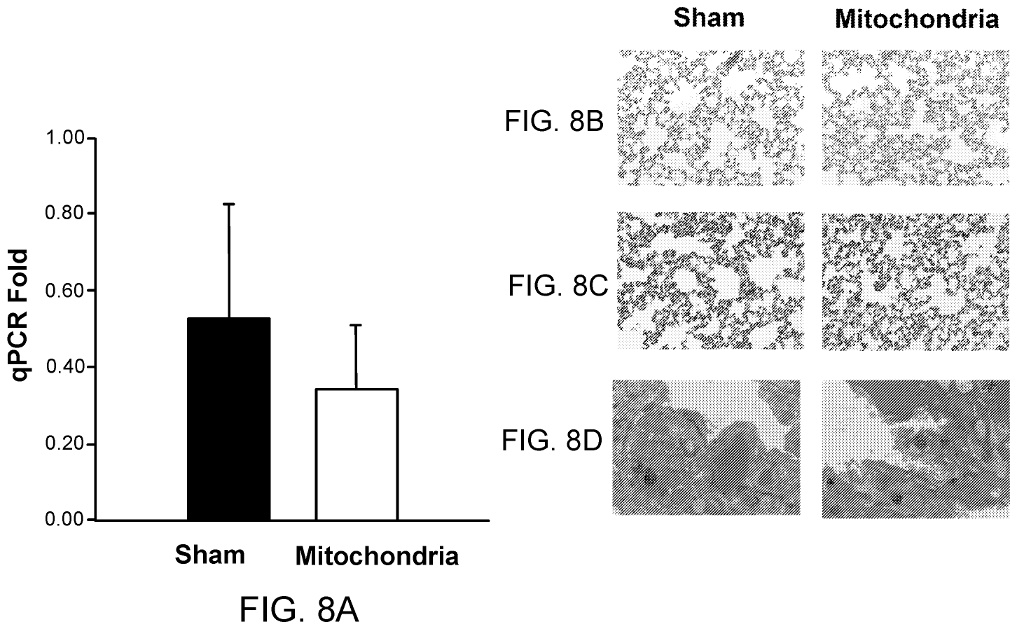

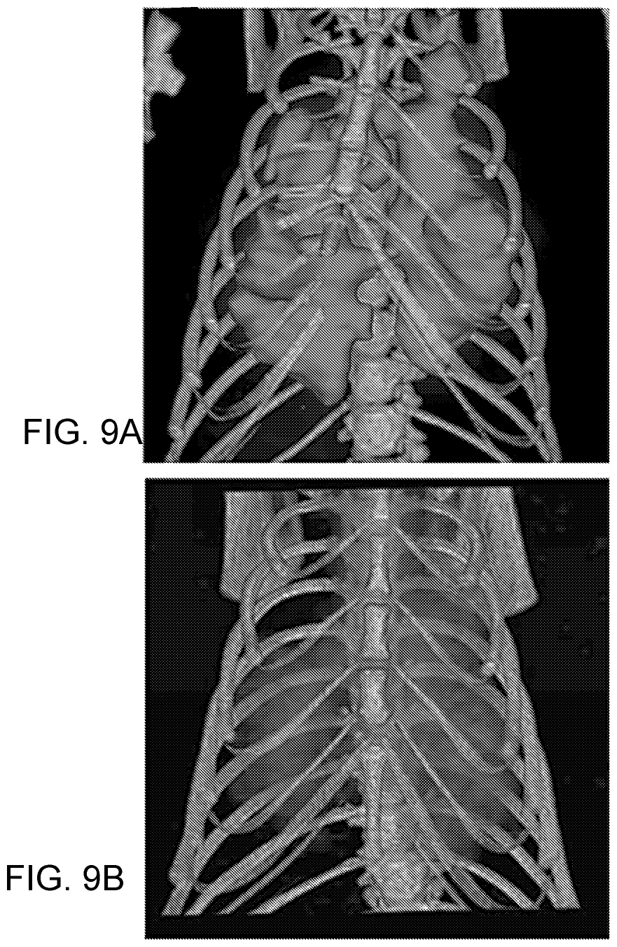

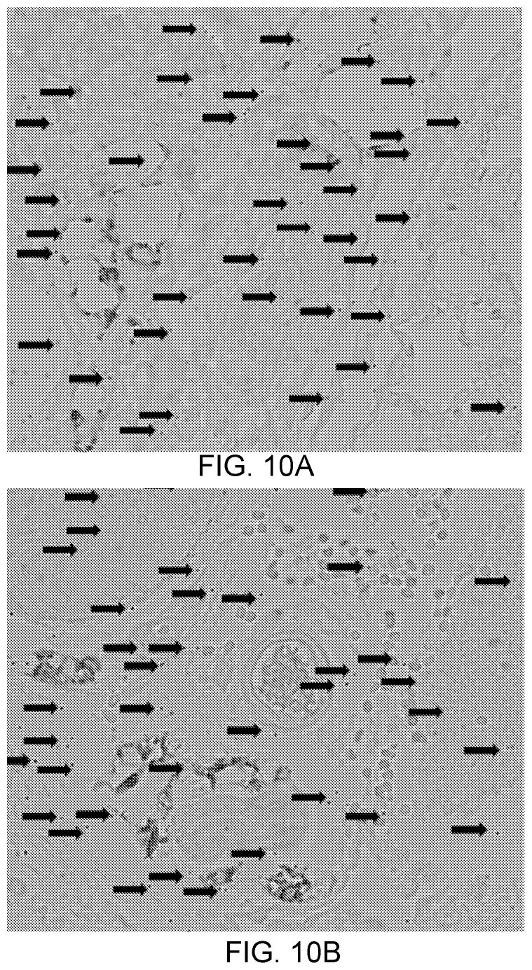

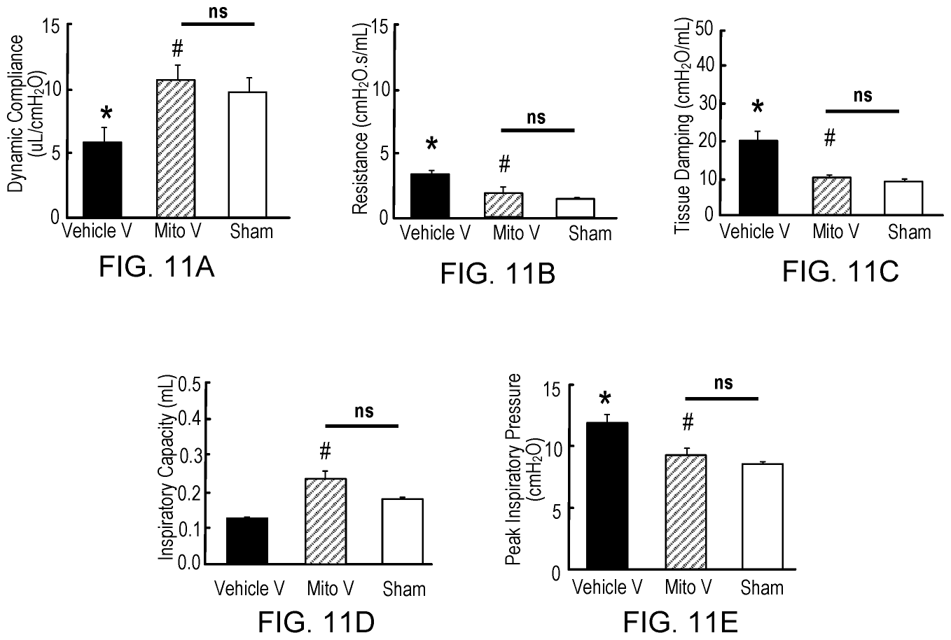

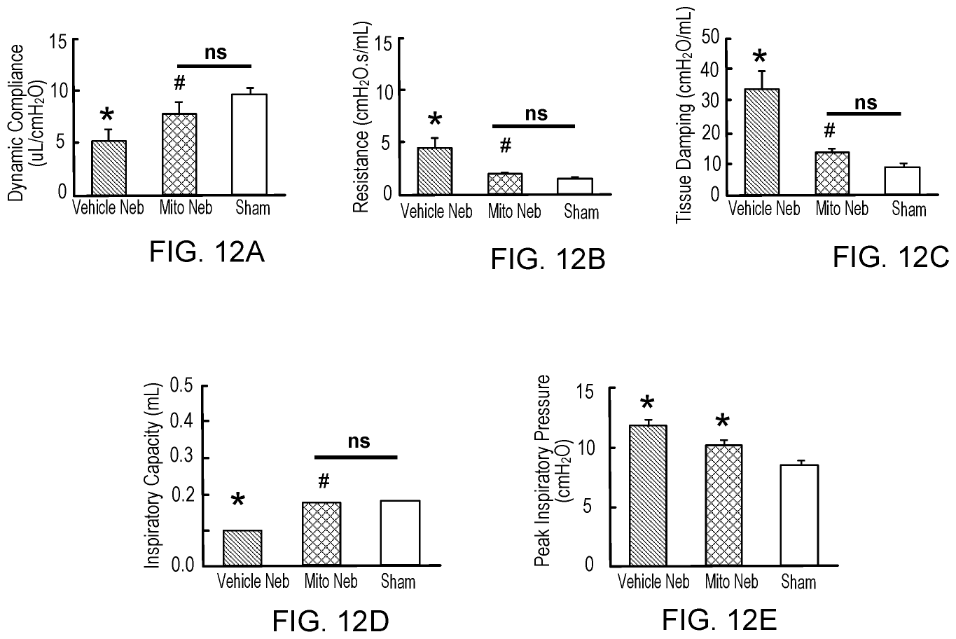

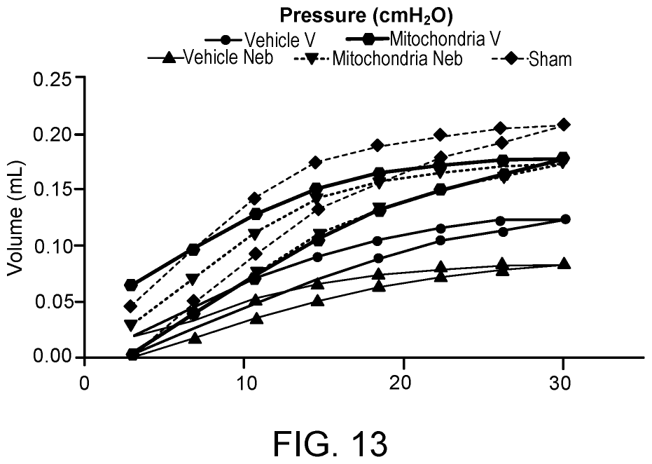

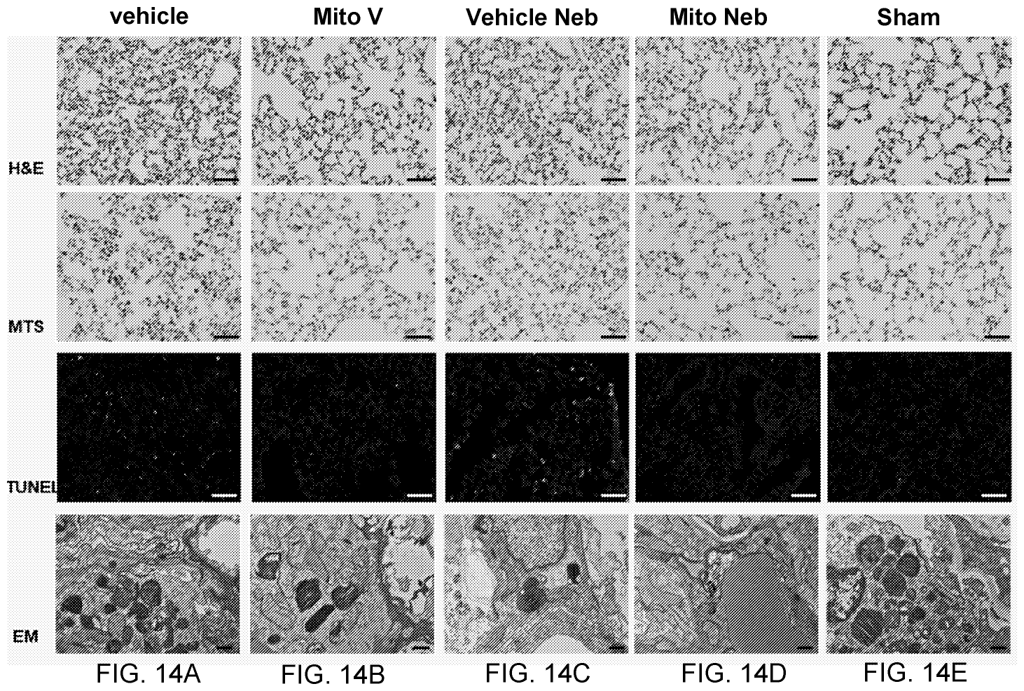

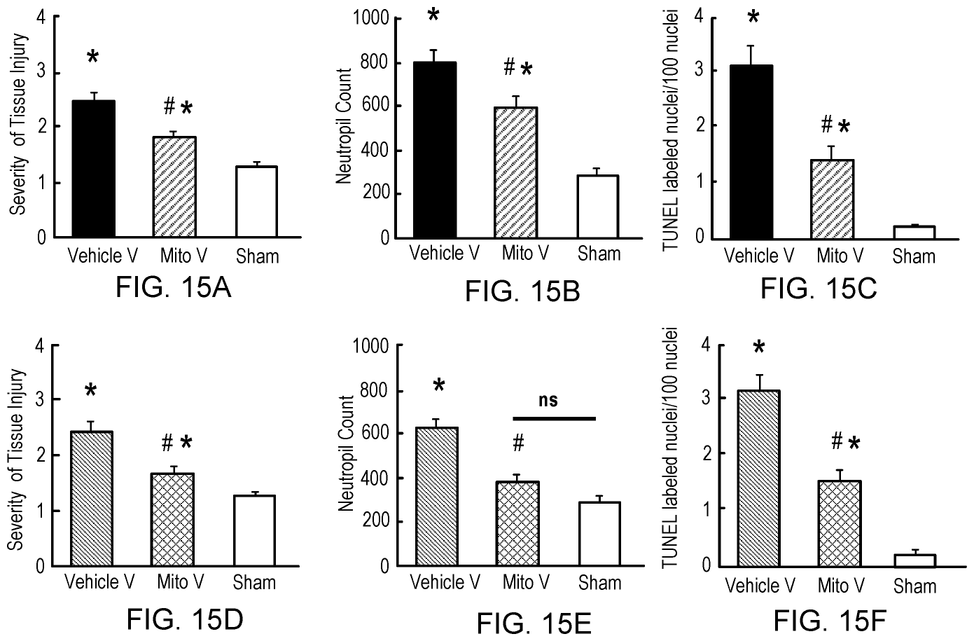

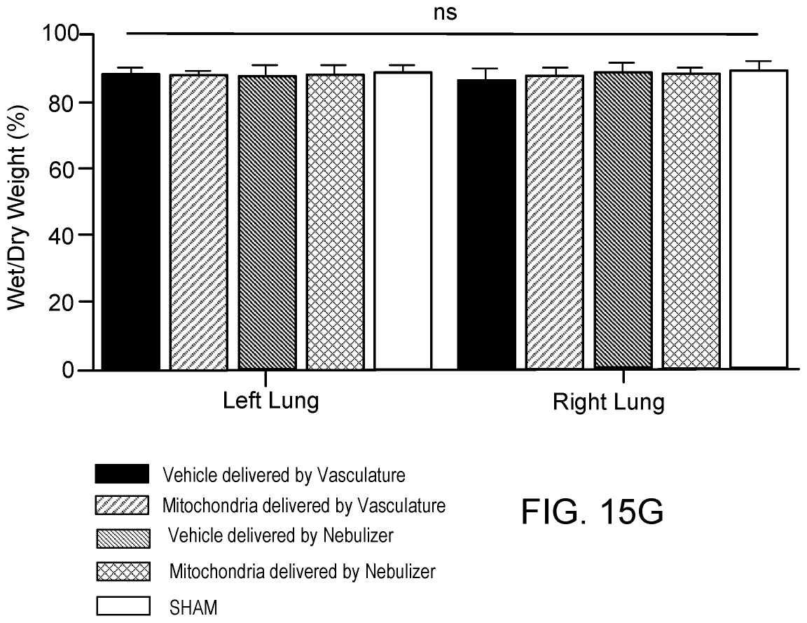

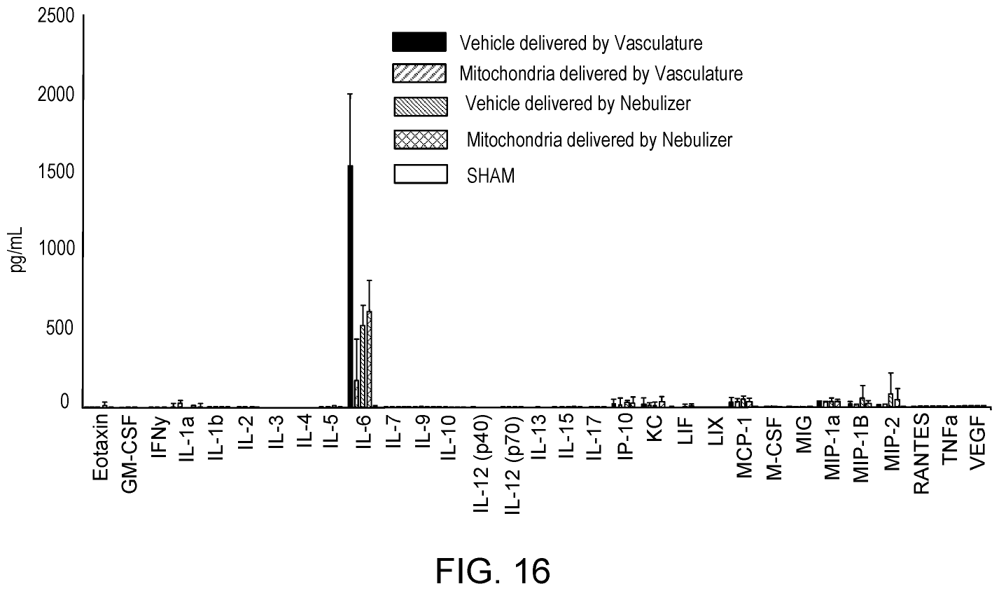

To allow for the isolation of viable respiration competent mitochondria, a rapid method was developed for the isolation and purification of mitochondria that can be performed in 20-30 minutes (FIG. 1). The major advantages of this method are: (1) it requires only a small amount of tissue that can be isolated using a #6 biopsy punch (<0.01 g tissue); (2) it uses a standardized tissue dissociation process that allows for uniform and consistent homogenization of tissue that is not easily achieved with manual homogenization methods; and (3) the use of differential filtration, rather than centrifugation, eliminates time-consuming and repetitive centrifugation steps and shortens the preparation time. In brief, two small pieces of skeletal muscle tissue (<0.1 gram) were obtained using a #6 biopsy punch. The tissue was homogenized in a volume of 5 mL of isolation buffer (300 mmol/L sucrose, 10 mmol/L HEPES-KOH, 1 mmol/L EGTA-KOH, pH 7.4) and then incubated for 10 minutes with Subtilisin A enzymatic on ice. The digested tissue was then filtered through a series of filters pre-wetted with isolation buffer, and the mitochondria were subsequently precipitated by centrifugation at 9×G for 5 minutes at 4° C.

### Results

1. A method of treating a subject having a respiratory disorder, the method comprising administering a composition comprising a therapeutically effective amount of mitochondria to the subject through respiratory tract.

2. The method of claim 1, wherein the composition is an aerosolized composition.

3. The method of claim 1, wherein the composition is converted into an aerosol form prior to administration to the subject by using a nebulizer, a vaporizer, a nasal sprayer, a pressurized metered dose inhaler, or a breath activated pressurized metered dose inhaler.

4. The method of claim 3, wherein the aerosol form of the composition comprises droplets that have a median size from 1 to 1000 microliters.

5. The method of claim 1, wherein the composition comprises respiration-competent mitochondria.

6. The method of claim 1, wherein the subject has acute lung injury, respiratory failure, reduced respiratory function, lung inflammation, lung carcinoma, skin wrinkles, baldness, and/or cancer.

7. (canceled)

8. The method of claim 1, wherein the concentration of mitochondria in the composition is about 1×10⁵ to 5×10⁸ ml⁻¹.

9. The method of claim 1, wherein the subject is administered about 1×10⁵ to 1×10⁹ of mitochondria per dose.

10. The method of claim 1, wherein the mitochondria are autogenic, allogeneic, or xenogeneic.

11. The method of claim 1, wherein the composition further comprises a solution selected from the group consisting of: K⁺-HEPES with a pH from 7 to 8, saline, phosphate-buffered saline (PBS), serum, and plasma.

12. The method of claim 1, wherein the composition further comprises one or more osmolytes selected from the group consisting of: trehalose, sucrose, mannose, glycine, proline, glycerol, mannitol, sorbitol, betaine, and sarcosine.

13. The method of claim 1, wherein the composition further comprises a pharmaceutical agent, a therapeutic agent, a diagnostic agent, a chemotherapeutic agent, and/or a pharmaceutically acceptable diluent, excipient, or carrier,
wherein the therapeutic agent or the diagnostic agent is linked to the mitochondria by a covalent bond, is embedded in the mitochondria, or is internalized within the mitochondria.

14.-19. (canceled)

20. The method of claim 1, wherein the mitochondria are genetically modified.

21. The method of claim 1, wherein the mitochondria comprise exogenous polypeptides, polynucleotides, DNA, RNA, mRNA, micro RNAs, nuclear RNAs, and/or siRNA.

22.-23. (canceled)

24. An aerosolized composition comprising
a plurality of liquid droplets comprising a buffer, wherein at least one liquid droplet in the plurality of liquid droplets comprises
at least one mitochondrion.

25. The aerosolized composition of claim 24, wherein the buffer is selected from the group consisting of: K+-HEPES with a pH from 7 to 8, saline, phosphate-buffered saline (PBS), serum, and plasma.

26. The aerosolized composition of claim 24, further comprising one or more osmolytes selected from the group consisting of: trehalose, sucrose, mannose, glycine, proline, glycerol, mannitol, sorbitol, betaine, and sarcosine.

27. The aerosolized composition of claim 24, wherein the composition further comprises a pharmaceutical agent, a therapeutic agent, a diagnostic agent, a chemotherapeutic agent, and/or a pharmaceutically acceptable diluent, excipient, or carrier,
wherein the therapeutic agent or the diagnostic agent is linked to the mitochondrion by a covalent bond, is embedded in the mitochondrion, or is internalized within the mitochondrion.

28.-38. (canceled)

39. The aerosolized composition of claim 24, wherein the composition comprises autogenic mitochondria, allogeneic mitochondria, xenogeneic mitochondria, or a mixture thereof.

40. (canceled)

41. A device for delivering mitochondria to a subject through respiratory tract, the device comprising:
a housing;
a reservoir disposed within the housing for a composition comprising mitochondria;
an aerosol generator to produce an aerosol form of the composition; and
an outlet through which the composition is delivered to the respiratory tract of the subject.

42.-46. (canceled)
